(19) United States
(12) Patent Application Publication
(10) Pub. No.: US 2014/0093884 A1
(43) Pub. Date: Apr. 3, 2014

(54) Process for Identifying Novel Anti-Inflammatory Molecules with Reduced Direct Transrepression of Genes Induced by Glucocorticoids

(71) Applicants: Pierre Chambon, Blaesheim (FR); Daniel Metzger, Strasbourg (FR); Milan Surjit, Illkirch (FR)

(72) Inventors: Pierre Chambon, Blaesheim (FR); Daniel Metzger, Strasbourg (FR); Milan Surjit, Illkirch (FR)

(73) Assignees: UNIVERSITE DE STRASBOURG, Strasbourg (FR); INSTITUT NATIONAL DE LA SANTE ET DE LA RECHERCHE MEDICALE (INSERM), Paris (FR); CENTRE NATIONAL DE LA RECHERCHE SCIENTIFIQUE (CNRS), Paris (FR)

(21) Appl. No.: 14/111,495

(22) PCT Filed: Apr. 13, 2012
(86) PCT No.: PCT/EP2012/056837

§ 371 Date: Nov. 26, 2013

Related U.S. Application Data
(60) Provisional application No. 61/475,445, filed on Apr. 14, 2011.

(30) Foreign Application Priority Data
Apr. 14, 2011 (EP) 11305447.2

(51) Int. Cl. G01N 33/50

(52) U.S. Cl. CPC G01N 33/5023
USPC 435/6.12; 435/29; 536/24.1; 435/320.1; 435/8; 435/6.13

(57) ABSTRACT

The present invention relates to a new process for identifying novel anti-inflammatory molecules with reduced direct transrepression of genes induced by glucocorticoids.

The inventors have discovered that GCs-mediated transrepression can be mediated not only via the tethering indirect pathway, but also through direct binding of GR to “simple” negative GREs (nGRE), which belongs to a novel family of evolutionary-conserved cis-acting negative response elements (IR nGREs), and are found in numerous GC-repressed genes.

### BACKGROUND OF THE INVENTION

Glucocorticoids (GCs) are peripheral effectors of circadian and stress-related homeostatic functions fundamental for survival throughout vertebrate life span (Chrousos, 2009; Nader et al., 2010). They are widely used to combat inflammatory and allergic disorders and their therapeutic effects have been mainly ascribed to their capacity to suppress the production of proinflammatory cytokines (Rhen and Cidlowski 2005). GCs act by binding to the GC receptor (GR), a member of the nuclear receptor (NR) superfamily. In absence of GCs, GR is maintained in the cytoplasm by molecular chaperones. Binding of GCs generates a conformational switch in the GR ligand binding domain (LBD) which affects GR interactions with chaperones and facilitates nuclear translocation (Ricketson et al., 2007). Once in the nucleus, GR binds to GC response elements (GREs) in the promoter region of the target genes, resulting in the regulation of their transcription (FIG. 1).

GREs are generally classified into “simple” GREs and “tethering” GREs. “Simple” GREs belong to a family of imperfect palindromes consisting of two inverted hexameric half-site motifs separated by 3 base pairs (bp) (Meijsing et al, 2009). Agonist-liganded GRs can bind directly to said palindromic DNA binding sites (DBS) for gene expression. On the contrary, “tethering” GREs do not contain DBS for GR per se, but instead contain binding sites for other DNA-bound regulators, such as NFκB and AP1, that recruit GR (Karin, 1998). Thus, “tethering” GREs confer “indirect” gene regulation to agonist liganded GR.

Many “simple” GREs have been identified so far. These GREs, also called “(+)GRE” confer direct transcriptional transactivation to agonist-liganded GR through association with co-activators (e.g. SRC1, TIF2/SRC2 and SRC3) (Lonard and O'Malley, 2007). The proteins encoded by these transactivated genes have a wide range of effects including for example regulation of gluconeogenesis.

“Simple” GREs are known to be linked to gene transactivation only. In contrast, “tethering” GREs are commonly associated with “tethered” indirect transrepression of target genes. These target genes are mainly pro-inflammatory genes, including interleukins, chemokines, and cytokines genes.

The anti-inflammatory properties of GCs represent the central target of pharmacological GC therapy. However, the physiological GCs and currently existing GC analogues do not distinguish among transactivation and transrepression, and control both the expression of “wanted” pro-inflammatory genes and of “unwanted” genes inducing diabetogenic activity, osteoporosis, as well as skin atrophy. Intensive research has been therefore carried out for discovering selectively acting (so-called dissociated) GCs that will be able to modulate the expression of the desirable genes only.

Glucocorticoids are also involved in glucose metabolism. The metabolic effects of these compounds are at least i) stimulation of gluconeogenesis (in particular, in the liver), ii) mobilization of amino acids from extrahepatic tissues, iii) inhibition of glucose uptake in muscle and adipose tissue, iv) stimulation of fat breakdown in adipose tissue. These effects are often leading to insulin resistance and to diabetes, or result in hypertension. Other effects have been observed, such as inhibition of bone formation, suppression of calcium absorption (both leading to osteoporosis), delayed wound healing, muscle weakness, and/or increased risk of infection.

It has been proposed that debilitating effects of GC treatment are mostly due to (+)GRE-mediated gene transactivation, while GC beneficial anti-inflammatory effects have been ascribed to “tethered” indirect transrepression (Karin, 1998; Kassel and Herrlich, 2007). This hypothesis has consequently led to a search for selective “dissociated” GR ligands which would preferentially generate “tethered” indirect transrepression, and not direct transactivation. Such a ligand, RU24858 (a selective GR agonist with steroid structure), was found to exhibit the expected dissociated profile in vitro (Vayssière et al., 1997). However, upon RU24858 administration in vivo, pathophysiological studies failed to confirm this dissociation (Belvisi et al., 2001).

The present Inventors have discovered that GCs-mediated transrepression can be mediated not only via the tethering indirect pathway, but also through direct binding of GR to “simple” negative GREs (nGRE), which belongs to a novel family of evolutionary-conserved cis-acting negative response elements (IR nGREs), and are found in numerous GC-repressed genes. Thus, GR agonists that induce indirect transrepression of pro-inflammatory genes, could also generate debilitating side effects via direct transrepression. Therefore, previous attempts, aimed at identifying GC analogs exhibiting a “dissociated” profile may have failed, because such GCs had kept their IR nGRE-mediated transrepression activity.

The herein presented results show that “ideal” GR agonists aimed at pharmacological therapy of inflammatory diseases should repress gene expression through “tethered” transrepression, while exhibiting reduced IR nGRE-mediated transrepression and (+)GRE-mediated transactivation activities. Thus, screening processes for identifying anti-inflammatory “dissociated” GCs which are mostly devoid of debilitating side effects should look for compounds that would exhibit decreased (+)GRE-mediated transactivation activity, as well as IR nGRE-mediated direct transrepression activity. The present Invention discloses such screening processes. The present Invention also discloses luciferase reporter plasmids that are useful for such screenings, as well as for characterizing the mode of action of some non-GC-derived compounds that may exhibit some of the beneficial therapeutic activities of “dissociated” (ICs, but be mostly devoid of their detrimental effects (De Bosscher and Haegeman, 2009).

### FIGURE LEGENDS

FIG. 1 represents the biological mechanisms underlying the process of the present invention, and highlights the three main pathways involved in GR-dependent gene regulation: 1) direct transactivation, 2) direct transrepression, and 3) tethered indirect transrepression.

FIG. 2 discloses the PGL4-AP1 vector of SEQ ID NO:55 which can be used in the in vitro process of the invention.

FIG. 3 discloses the PGL4-NFκB vector of SEQ ID NO:56 which can be used in the in vitro process of the invention.

FIG. 4 discloses the PGL3(VDRE) IR1 nGRE vector of SEQ ID NO:50, which can be used in the in vitro process of the invention.

FIG. 5 discloses the PGL3 (VDRE) (+) GRE vector of SEQ ID NO:54 which can be used in the in vitro process of the invention.

FIG. 6 discloses the sequence from position 1-656 of PGL3 (VDRE) IR0 nGRE vector of SEQ ID NO:51 which can be used in the in vitro process of the invention.

FIG. 7 discloses the sequence from position 1-658 of PGL3 (VDRE) IR2 nGRE vector of SEQ ID NO:52 which can be used in the in vitro process of the invention.

FIG. 8 discloses the PGL3 (SV40/VDRE) IR1 nGRE vector of SEQ ID NO:49 which can be used in the in vitro process of the invention.

FIG. 9 discloses the PGL3 (SV40/VDRE) (+) GRE vector of SEQ ID NO:52 which can be used in the in vitro process of the invention.

FIG. 10 discloses some of the experimental results obtained by the present Inventors and presented in the experimental part below. The transcript levels of TSLP, CYP26A1 and GPX3 are determined in ear epidermis after a 6 hrs topical treatment with the GR agonist FA, the GR antagonist RU486, the low calcemic Vitamin D3 analog MC903, alone or in combination, as indicated.

FIG. 11 discloses some of the experimental results obtained by the present Inventors and presented in the experimental part below. Luciferase activity in A549 cells transfected with VDRE reporter plasmid (a derivative of PGL3(VDRE) IR nGRE vector in which the IR nGRE element is deleted), PGL3(+)GRE (a derivative of PGL3(VDRE) (+)GRE vector in which the VDRE element is deleted), PGL3 IR1 nGRE (a derivative of PGL3(VDRE) IR nGRE vector in which the VDRE element is deleted), PGL3(VDRE/(+)GRE (of SEQ ID NO 54; FIG. 5), PGL3(VDRE/IR1 nGRE (of SEQ ID NO 50; FIG. 4) and treated with FA and/or Vitamin D3 (VD3), as indicated.

FIG. 12 discloses some of the experimental results obtained by the present Inventors and presented in the experimental part below, showing that IR nGREs of mouse and human orthologues may differ by a “tolerable” one base pair mutation.

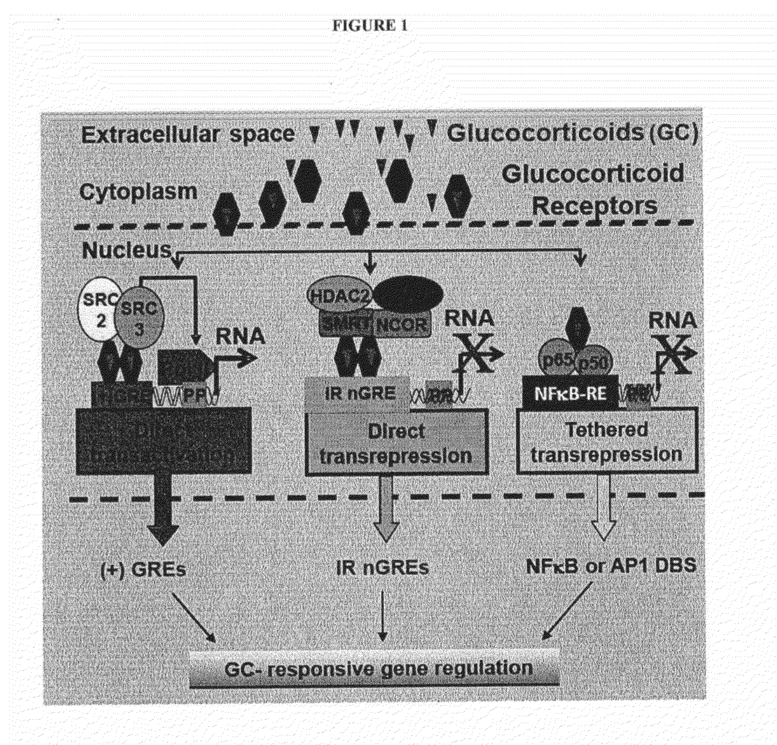

(A) Sequence and position (from the +1 transcription start site) of nGRE motifs present in human (h) and mouse (m) Keratin 5 (K5) gene. Bold letters in low case denote non canonical bases in IR1 nGREs. nGRE regions that assemble a GR and corepressor complex upon Dex-treatment is boxed.
(B) Sequence and position (from the +1 transcription start site) of nGRE motifs present in human (h) and mouse (m) insulin receptor (insr) and insulin (ins) genes. Bold letters in low case denote non-canonical bases in IR1 nGREs. nGRE regions that assemble a GR and corepressor complex upon Dex-treatment is boxed.
(C) Sequence and position (from the +1 transcription start site) of nGRE motifs present in human (h) and mouse (m) Reverbα gene. Bold letters in low case denote non canonical bases in IR1 nGREs. nGRE regions that assemble a GR and corepressor complex upon Dex-treatment is boxed.
(D) ChIP analysis of epidermis, pancreas and liver showing the binding of GR and corepressors to the IR nGRE regions of indicated genes. WT mice were topically-treated with vehicle or Dex (in the case of K5) or IP-injected with vehicle or Dex (in the case of ins, insr and Reverbα) for 18 hours.

FIG. 13 discloses some of the experimental results obtained by the present inventors and presented in the experimental part below, showing that FA induces repression of reporter genes containing the IR1 nGRE DBS of various genes irrespective of their CC-induced repression in vivo.

Luciferase assays on A549 cells transfected with various PGL3(VDRE) IR1 nGRE vectors containing the IR1 nGREs present in different genes (Table 4) as indicated, were treated for 6 hours with vehicle, VD3 and/or FA, as indicated.

FIG. 14 discloses some of the experimental results obtained by the present Inventors and presented in the experimental part below, showing differential effects of GRdim mutation and RU486 treatment on “tethered”- and IR nGRE-mediated transrepression. Gene transcripts in epidermis of WT and GRdim mutant mice topically treated as indicated for 6 hours are analysed by Q-RT-PCR.

FIG. 15 discloses some of the experimental results obtained by the present Inventors and presented in the experimental part below, showing that:
A) Dexamethasone and RU24858 similarly repress NFκB and AP1-driven transcription in vitro. A549 cells transfected with PGL4-NFκB (NFκBluc plasmid) of SEQ ID NO:56 and PGL4-AP1 (AP1luc plasmid) of SEQ ID NO:55 were treated as indicated for 6 hours, followed by luciferase assay. “BAY”=NFκB-specific inhibitor BAY 11-7082 and “JNK inhibit”=JNK inhibitor II).
B) Q-RT-PCR of gene transcripts from WT dorsal epidermis topically-treated with Dexamethasone or RU24858 for 18 hours (left panel) and from liver of WT mice IP-injected with Dexamethasone or RU24858 for 18 hours (right panel).

### DETAILED DESCRIPTION OF THE INVENTION

The present inventors have discovered a widespread conserved family of “negative” palindromic GC-response elements (IR nGREs) that mediate transrepression by direct binding of GC-agonist-liganded GR. GC-induced IR nGRE-mediated direct transrepression is distinct from GC-induced “tethered” indirect transrepression, as: (i) “tethering” GREs do not contain DNA binding sites for GR per se, but instead binding sites for other DNA-bound transregulators (e.g. NFκB and AP1) that recruit GR, (ii) the GRdim mutation that does not affect “tethered” transrepression, abolishes IRnGRE-mediated direct transrepression, (iii) co-treatment with RU486 relieves GC-induced IRnGRE-mediated direct transrepression, whereas “tethered” transrepression is not or only slightly affected.

Investigating in vitro whether the integrity of the TSLP (Thymic Stromal Lymphopoietin) nGRE in which a 1 bp spacer separates the Inverted Repeated motifs (IR1 nGRE) is essential for its function, has shown that no (IR0 nGRE) or a 2 bp (IR2 nGRE) spacer is “tolerable”. Moreover, one base changes at any position of the “canonical” repeated motifs of the TSLP IR1 nGRE and of its IR2 nGRE derivative is, with one exception, functionally “tolerable” in vitro. Mouse and human genome-wide analyses revealed the presence of hundreds of mouse and human orthologue genes containing conserved canonical IR0, IR1 and IR2 nGREs (see Table 1, below). In no case are these nGREs located in the near vicinity (<100 bp) of binding sites for regulatory factors (i.e. there is no evidence that IR nGREs are composite sites), and most of them are conserved throughout vertebrates (mammals, chicken and zebra fish, our unpublished data). A number of “IR0”, “IR1” and “IR2” nGRE-containing genes expressed in mouse epidermis, intestinal epithelial cells or liver, were analyzed for (i) repression by the GC-agonist Dexamethasone (Dex)-treatment, (ii) prevention of this repression by the GC antagonist RU486 cotreatment, (iii) association of their IR nGREs with agonist liganded-GR and corepressors (ChIP assays), and (iv) repressing activity of their nGREs in vitro, which taken all together represent the signature of IR nGRE-mediated transrepression. This analysis demonstrates that IR0, IR1 and IR2 nGRE-containing genes can be efficiently transrepressed by agonist-liganded GRs bound to their nGREs together with corepressors. Assuming that the mouse/human genes that have been randomly selected are representative, the data of the present Inventors indicate that the expression of approximately 600 mouse/human orthologue genes could be negatively controlled through nGRE-mediated transrepression in epidermis, intestinal epithelium and liver. Therefore provided the remaining 400 mouse/human orthologue genes that contain canonical IR nGREs are expressed in other tissues, it is likely that the expression of at least 1000 orthologue genes are negatively controlled in the mouse by GC-induced IR nGRE-mediated transrepression. In fact, the actual number of genes whose expression could be negatively controlled by GC-induced IR nGRE mediated transrepression might be much higher, as the existence of functionally “tolerable” single base changes in IR nGREs of human and/or mouse orthologue genes could result in underestimating by several hundreds the actual number of genes containing functional IR nGREs.

GCs that act as end-effectors of the HPA (hypothalamus-pituitary-adrenal) axis, are secreted by adrenal glands in a circadian and stress-related manner. They influence the functions of virtually all organs and tissues throughout life span, and are essential for maintenance of their homeostasis and important biological activities, such as intermediary metabolism, immune and inflammatory reactions, as well as circadian clock and stress systems (Chrousos, 2009; Nader et al., 2010). Our ontology search has revealed that a number of IR nGRE-containing genes are involved in such functions. We focus here on a few examples illustrated in the present study.

The data below unequivocally demonstrate that GC-induced IR nGRE-mediated repressions of both the insulin precursor gene (in pancreas β cells) and insulin receptor gene (in liver) are early stress-induced events. This ensures that, upon occurrence of a stress, an elevation of blood glucose level will rapidly follow the surge of GC secretion, thereby providing the increased nutrition of brain, heart and skeletal muscles, required for the central coordination of stress response (Chrousos, 2009). On the other hand, under conditions of chronic stress, GC-induced IR nGRE-mediated repression of the insulin receptor gene may cause insulin resistance and lead to diabetes. This study also reveals the existence of functional IR1 nGREs in ACTH receptor (melacortin 2 receptor, MC2R) gene, and ACTH receptor accessory protein (MRAP) gene, thereby adding, at the adrenal level, another step to the closed negative feedback loop that resets the HPA axis by regulating the synthesis of secreted GCs through GR-mediated repression of CRH and POMC gene expression in hypothalamus and pituitary, respectively (Dostert and Heinzel, 2004). The GC-induced IR nGRE-mediated repression for the Reverbα gene and the RORα gene were also found, both intervening in the control of the circadian timing system, and being likely to play an important role in communications of the molecular Clock and stress systems with intermediary metabolism, which are fundamental for survival (Duez and Staels, 2008; Nader et al., 2010; and Refs therein).

Importantly, it was known from previous studies that the expression of a number of genes is decreased upon GC therapy, but the underlying pathophysiological molecular mechanisms remained often unknown. The data below demonstrate that, upon GC therapy, decreases of keratins 5 and 14 in skin, as well as those in Cyclin D1 and CDK4, are due to GC-induced IR nGRE-mediated repression. Hsd11β2 is another important gene that is repressed by IR1 nGRE-mediated GC-treatment in both skin and colon tissues. The 11β-HSD2 enzyme (encoded in Hsd11β2 gene) is responsible for inactivating glucocorticoids in mineralocorticoid receptor (MR) target tissues (Gross and Cidlowski, 2008). In the absence of this enzyme, GCs (corticosterone in mice) activate MR despite the absence of aldosterone, resulting in hypertension (Stewart et al., 1996). That Hsd11β2 is an IR1 nGRE-containing gene provides a possible molecular explanation for GC therapy-induced hypertension.

Thus, the herein presented results show that a molecule exhibiting, via GR-dependent tethered indirect transrepression, anti-inflammatory activities of GC, while significantly lacking their unwanted side effects, would be most useful. Such a molecule should be low in IR nGRE-mediated transrepression and (+)GRE-mediated transactivation activities.

The present Invention discloses screening processes enabling to identify such molecules. These processes can be performed either with cultured GR-containing cells by testing the expression of reporter genes or the expression of genomic genes which are modulated by GC, as well as by testing the expression of GC-modulated genomic genes in tissues of laboratory animals.

In a first aspect, the present invention discloses a process for selecting a molecule exhibiting the anti-inflammatory activities of glucocorticoids (GCs), and having reduced GC-dependent IR nGRE-mediated direct transrepression activity, comprising at least the step of testing if a candidate molecule:
a) significantly transrepresses the transcription of at least one gene containing among its promoter elements NFκB or AP1 DNA binding sequence (DBS),
b) significantly transrepresses the transcription of at least one gene containing among its promoter elements an IR-type negative element (IR nGRE DBS),
c) significantly activates the transcription of at least one gene containing among its promoter elements a (+) GRE DBS,

wherein said IR nGRE DBS is chosen in the group consisting of: sequence SEQ ID NO:1 (IR1 nGRE), SEQ ID NO:2 (IR0 nGRE), SEQ ID NO:3 (IR2 nGRE) and tolerable variants thereof,

wherein said NFκB DBS has the sequence SEQ ID NO: 4, SEQ ID NO:5 or SEQ ID NO:6 or variants thereof,

wherein said AP1 DBS has the sequence SEQ ID NO:7, SEQ ID NO:8 or variants thereof, and

wherein said (+) GRE DBS has the sequence SEQ ID NO:9 or SEQ ID NO:10 or variants thereof.

Said candidate molecule will be selected:
if it significantly transrepresses the transcription of said gene in step a), but it does not significantly transrepress the transcription of said gene in step b), or
if it significantly transrepresses the transcription of said gene in step a), but does not significantly transrepress the transcription of said gene in step b) and does not significantly increase the transactivation of the transcription of said gene in step c).

More precisely, the present invention targets a process for selecting a molecule exhibiting, via glucocorticoid receptor (GR)-dependent tethered indirect transrepression, the anti-inflammatory activities of GCs, and having reduced GC-dependent IR nGRE-mediated direct transrepression activity, comprising at least the following steps:

i) providing at least one candidate molecule,

ii) testing if said candidate molecule:
a) significantly induces GR-dependent tethered indirect transrepression of the transcription of at least one gene containing among its promoter elements at least one NFκB or one AP1 DNA binding sequence (DBS),
b) significantly induces GR-dependent direct transrepression of the transcription of at least one gene containing among its promoter elements an IR-type negative element nGRE DBS),
c) significantly induces GR-dependent transactivation of the transcription of at least one gene containing among its promoter elements a (+) GRE DBS,

iii) selecting said candidate molecule as a molecule exhibiting anti-inflammatory activities of GCs:
if it significantly transrepresses the transcription of said gene in step a), but it does not significantly transrepress the transcription of said gene in step b), or
if it significantly transrepresses the transcription of said gene in step a), but does not significantly transrepress the transcription of said gene in step b) and does not significantly increase the transactivation of the transcription of said gene in step c),

wherein said IR nGRE DBS, NFκB DBS, AP1 DBS and (+) GRE DBS are as defined above.

In a particular embodiment of the invention, the candidate molecule which is selected in step iii) of the process of the invention significantly transrepresses the transcription of said gene in step a), but does not significantly transrepress the transcription of said gene in step b) and does not significantly transactivate the transcription of said gene in step c).

It should be noted that, as used in the present application, the expressions “transactivation of a gene”, “transactivation of the transcription of a gene” and “transactivation of the expression of a gene” are equivalent. Similarly, the expressions “transrepression of a gene”, “transrepression of the transcription of a gene” and “transrepression of the expression of a gene” are equivalent.

The process of the invention enables to screen any natural or synthetic compound which can be obtained (such compound will be hereafter referred to as a “candidate molecule”). It can be for example a GR ligand, a GC, a GC analogue, as well as any non-GC-derived molecule. GC analogues are defined as synthetic molecules exhibiting some or all of the physiological activities of GCs (Cortisone in humans, and corticosterone in rodents).

As used herein, the term “glucocorticoïd” (GC) relates to a class of steroid hormones that bind to the glucocorticoid receptor (GR). These GC therefore contains a specific arrangement of four cycloalkane rings that are joined to each other. They can be natural or synthetic. Known GCs are for example hydrocortisone (cortisol), prednisone, prednisolone, methylprednisolone, dexamethasone (Dex), fluocinolone acetonide (FA), betamethasone, triamcinolone, beclometasone, fludrocortisone, deoxycorticosterone, aldosterone, etc. These compounds are known to mediate effects of two major categories: immunological and metabolic. Through their interaction with the glucocorticoid receptor, they can:
up-regulate the expression of anti-inflammatory proteins, and
down-regulate the expression of pro-inflammatory proteins.
They are also shown to play a key role in the development and homeostasis of T lymphocytes (Rhen and Cidlowski 2005).

As used herein, a molecule “exhibits the anti-inflammatory activity of glucocorticoids (GCs)” when it induces similar regulation of the expression of anti-inflammatory proteins, or of pro-inflammatory proteins, as compared with known GCs such as hydrocortisone or dexamethasone. This regulation can be demonstrated in vitro at the protein level or at the mRNA level, by Q-RT-PCR assay. This similar regulation can also be observed in vivo, by the same assays.

As used herein, the term “having reduced GC-dependent IR nGRE-mediated direct transrepression activity” designates a reduction of this repression activity of at least 30%, preferably at least 60%, preferably at least 80% and more preferably 90%, in comparison with the reduction obtained with a glucocorticoid, preferably Dex, at the same dosage.

When used in the present application, the term “similar” means that the two compared phenomena are identical at least at 80%, preferably at least 85%, preferably at least at 90%, and more preferably at least at 95%. For example, two expression levels of a gene will be similar if the ratio of their mRNA levels (and/or their protein levels) is comprised between 0.80 and 1.25, preferably between 0.85 and 1.17, preferably between 0.9 and 1.1, and more preferably between 0.95 and 1.05.

A gene regulation is “glucocorticoid receptor (GR)-dependent” if it requires a functional GR to be effective. The person skilled in the art knows how to measure this property, for example by assessing in GR KO mice if said gene regulation can occur, or by blocking GR with an antagonist.

Genes containing a NFκB or AP1 DNA binding sequence (DBS) among their promoter elements and down-regulated by GC administration, have been already described. For example, such genes can be COX2, MMP13, IL4, and IL10 genes (see Tables 2 and 3, below).

Genes containing a (+)GRE DBS among their promoter elements and are upregulated by GC administration have also been already described. For example, such genes can be the GILZ, GPX3, GGT1 and ERP27 genes (see Tables 2 and 3, below).

Genes containing an IR-type negative element (IR nGRE DBS) among their promoter elements have been identified for the first time by the present Inventors (see Table 1-3 below). In a preferred embodiment, such genes are chosen in the group consisting of the IR0 nGRE, IR1 nGRE and IR2 nGRE-containing genes disclosed in Tables 2 and 3. Preferably, such genes can be BCL3, BHLHB2, ENC1, FFGFR3, FOXa2, GEM, MAFK CCND1, CYP26A1, JUND, HSD11β2, PRKCB, K14, RDH11, STRA13, RORα and TNFRSF19 genes.

TABLE 1
Genes containing IR0 nGRE, IR1 nGRE and IR2 nGRE DBS in their
promoter

| | | IR2 nGRE |
|---|---|---|
| IR0 nGRE | IR1 nGRE | containing |
| containing Genes | containing genes | genes |
| ADAMTS19 | ADRA2A | CCR10 |
| AIP | AGPAT2 | COL4A3BP |
| ANO4 | AKAP6 | DPAGT1 |
| CCND1 | ARHGAP23 | ELMOD3 |
| CITED1 | ATN1 | ERP27 |
| DDI1 | BCL2L1 | FKBP2 |
| DLAT | BEST2 | FLOT2 |
| FBXL18 | BMP3 | FOXJ1 |
| FSD2 | BTG2 | FOXQ1 |
| GPIHBP1 | B4GALT2 | FSTL1 |
| HCST | CACNA2D3 | GAS6 |
| INHBE | CADM4 | GAS2L2 |
| KLF5 | CDH11 | GGT1 |
| LAG3 | CDK9 | GIT2 |
| MAPK4 | CTSA | GKN1 |
| MTG1 | CYP26A1 | GLU1 |
| N MYC | DYNLT3 | GOT1 |
| NDUFA4L2 | FBLN2 | GPR68 |
| NEU1 | GBA2 | GPR97 |
| PPIL5 | GNG10 | GRASP |
| PTMS | GPR108 | GUK1 |
| SLC25A5 | GPR153 | HSPB2 |
| SP9 | HSD11B2 | HSPB3 |
| SPARC | JUND | ISL2 |
| TCF25 | KLC3 | ITPKA |
| TRPM7 | KRT14 | MUC20 |
| ZBED4 | LRRC | POMGNT1 |
| ZFP113 | MT2 | PRKRA |
| ZFP467 | MTHFR | RDH5 |
| ZFP784 | MX11 | RORA |
| | NKRF | SLC25A34 |
| | PRKCB | SLC2A8 |
| | RDH11 | SOCS1 |
| | SCP2 | TGM6 |
| | SGK3 | TNFRSF25 |
| | SOCS3 | |
| | SREBF1 | |
| | SSTR4 | |
| | STAB1 | |
| | STRA13 | |
| | TNFRSF19 | |
| | USF1 | |

The expression of all these NFκB or AP1 DNA DBS and IR nGRE DBS-containing genes are known to be decreased upon GC treatment.

All of these NFκB or AP1 DNA DBS, (+) GRE and IR nGRE DBS-containing genes are hereafter designated as “GC-dependent genes”.

The term “tethered indirect transrepression” encompasses the repression of a gene via the binding of agonist-GR bound to a transactivator (e.g. NFκB, AP1, . . . ) interacting with a cognate DBS.

The term “direct transrepression” encompasses the repression of a gene via the direct binding of agonist-liganded GR to an IR nGRE DBS.

The term “direct transactivation” encompasses the transactivation of a gene via the binding of agonist-liganded GR to a (+)GRE DBS.

As used therein, the expression of a gene is “significantly transrepressed” in presence of a candidate molecule if the level of expression of said gene in presence of said candidate molecule is decreased by a factor of at least two, preferably at least three, and more preferably of at least four as compared to the level of expression of said gene in absence of said candidate molecule. In other words, the expression level of said gene in the presence of said candidate molecule is of maximally 60%, preferably 30%, and more preferably 25% of the expression level of said gene in the absence of said candidate molecule. In this case, the expression level of said gene in the presence of said candidate molecule is also said to be “significantly lower” than the expression level of the gene in the absence of said candidate molecule.

On the contrary, the transcription of a gene is “significantly transactivated” in presence of a candidate molecule if the level of expression of said gene in presence of said candidate molecule is increased by a factor of at least two, preferably at least three, and more preferably of at least four as compared to the level of expression of said gene in absence of said candidate molecule. In other words, the expression level of said gene in the presence of said candidate molecule is of minimally 200%, preferably 300%, and more preferably 400% of the expression level of said gene in the absence of said candidate molecule. In this case, the expression level of said gene in the presence of said candidate molecule is also said to be “significantly upper” than the expression level of the gene in the absence of said candidate molecule.

Said gene expression level can be detected by any mean known by the person skilled in the art, for example by quantifying the mRNA level of said gene by RT-PCR and/or QRT-PCR, northern Blot, RNA micro-arrays, or any other RNA detecting mean. Said gene expression level can also be detected by measuring the amount of the gene-encoded protein by Elisa test, western blot, ImmunoHistochemistry, or HPLC or any other protein detecting mean.

As previously mentioned, the term “IR nGRE DBS” corresponds to a IR-type negative DNA binding site (DBS) which is present in the promoter of particular GR-responding genes (such as those listed in Table 1). This DBS is an imperfect palindromic sequence chosen in the group consisting of: CTCCxGGAGA with x=A, T, G or C (SEQ ID NO:1, also called “IR1 nGRE”), CTCCGGAGA (SEQ ID NO:2, also called IR0 nGRE), and CTCCxyGGAGA with x=A, T, G or C and y=A, T, G or C independently of each other (SEQ ID NO:3, also called IR2 nGRE) and tolerable variants thereof.

The present Inventors have found that various IR nGRE variants having a one base pair change as compared with said IR nGRE DBS, still enable GR-dependent direct transrepression. In a preferred embodiment, the tolerable variant of the IR nGRE DBS is thus derived by at least one base pair change from the sequence SEQ ID NO: 1 (IR1 nGRE), SEQ ID NO:2 (IR0 nGRE), or SEQ ID NO:3 (IR2 nGRE), and has a GR-dependent direct transrepression activity.

More precisely, said tolerable variant of SEQ ID NO:1 (IR1 nGRE) can be chosen in the group consisting of: SEQ ID NO:11 to 27, and said tolerable variant of SEQ ID NO:3 (IR2 nGRE) can be chosen in the group consisting of: SEQ ID NO:28 to 48.

The process of the invention involves genes containing, among their promoter elements, a NFκB DBS having a sequence SEQ ID NO: 4, SEQ ID NO:5 or SEQ ID NO:6, or variants thereof. Preferably, said variants have a sequence which is homologous to SEQ ID NO: 4, SEQ ID NO:5 or SEQ ID NO:6.

The process of the invention involves genes containing, among their promoter elements, a AP1 DBS having a sequence SEQ ID NO:7, SEQ ID NO:8 or variants thereof. Preferably, said variants have a sequence which is homologous to SEQ ID NO:7 or SEQ ID NO:8.

The process of the invention involves genes containing, among their promoter elements, a (+) GRE DBS having a sequence SEQ ID NO:9, SEQ ID NO:10 or variants thereof. Preferably, said variants have a sequence which is homologous to SEQ ID NO:9 or SEQ ID NO:10.

As used herein, the term “homologous” refers to sequences that have sequence similarity. The term “sequence similarity”, in all its grammatical forms, refers to the degree of identity or correspondence between nucleic acid sequences. In the context of the invention, two nucleic acid sequences are “homologous” when at least about 80%, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, alternatively at least about 99% of nucleic acids are similar. Preferably the similar or homologous nucleic acid sequences are identified by alignment using, for example, the GCG (Genetics Computer Group, Program Manual for the GCG Package, Version 7, Madison Wis.) pileup program, or using any of the proposed programs and algorithms dedicated thereto (for example, BLAST, or FASTA).

In a particular embodiment, the process of the invention comprises at least the following in vitro steps in cultured GR-expressing cells:
a) providing at least one candidate molecule,
b) providing a first vector comprising at least one NFκB or one AP1 DBS operatively coupled to a reporter gene and a promoter thereof active in said cell,
c) providing a second vector comprising an IR nGRE DBS operatively coupled to a reporter gene and a promoter thereof active in said cell,
d) providing a third vector comprising a (+) GRE DBS sequence operatively coupled to a reporter gene and a promoter thereof active in said cell,
e) selecting conditions in step b) wherein in NFκB and/or an AP1 factors are activated,
f) selecting conditions wherein said reporter genes of steps b) and c) are significantly expressed and significantly repressed by GCs, and wherein the expression of said reporter gene of step d) is significantly increased by the addition of GC,
g) measuring in said cells the level of expression of each reporter gene in the presence and absence of said candidate molecule, and under conditions selected in steps e) and f),
h) selecting said candidate molecule:
   if it significantly transrepresses the expression of said reporter gene of step b), but does not significantly transrepress the expression of said reporter gene of step c), or
   if it significantly transrepresses the expression of said reporter gene of step b), but does not significantly transrepress the expression of said reporter gene of step c), and does not significantly increase the expression of said reporter gene of step d).

As used herein, the term “vector” means the vehicle by which a DNA or RNA sequence of a foreign reporter gene can be introduced into an host cell so as to transform it and promote expression of the introduced reporter gene sequence. Vectors may include for example, plasmids, cosmids, YACs, phages, and viruses. For the purpose of the screening process of the invention, such vectors are preferably plasmids.

As used herein, the term “promoter” is a sequence of nucleotides from which transcription may be initiated of DNA operably linked downstream (i.e. in the 3′ direction on the sense strand of double stranded DNA). Within the promoter sequence will be found a transcription initiation site (conveniently found, for example, by mapping with nuclease S1), as well as protein binding domains (consensus sequences) responsible for the binding of RNA polymerase. Promoters which may be used to control the reporter gene expression in the context of the present invention are for example: the SV40 early promoter and retroviral LTRs, to name few. Other suitable promoters are well known from the person skilled in the art. Preferably, the promoter which is used in the vector of the invention is the enhancer-less SV40 early promoter coupled to the SV40 enhancer and/or the vitamin D3 response element (VDRE).

The vectors which are used in the process of the invention can also contain sites for transcription initiation, termination, and, in the transcribed region, a ribosome binding site for translation.

The reporter gene sequence is “operatively coupled with” an expression control sequence (for example a promoter) in a vector, when RNA polymerase is able, in define conditions, to transcribe the reporter sequence into RNA, which is then trans-RNA spliced (if it contains introns) and is translated into that reporter protein.

Such reporter protein can be any protein having a quantifiable expression level. It is preferably chosen from the group consisting of: luciferase, β-galactosidase and fluorescent proteins.

The person skilled in the art knows will easily define the appropriate conditions wherein the reporter genes of said first and second vectors will be expressed, especially in the absence of said candidate molecule. For example, the vectors can be transfected into appropriate host cells, in appropriate culture mediums.

Representative examples of appropriate hosts include, but are not limited to animal cells such as CV1, CHO, COS-1, 293 and A549 cells. Appropriate culture mediums and conditions for the above-described host cells are known in the art. Among preferred eukaryotic vectors are pWLNEO, pSV2CAT, pOG44, pXT1 and pSG available from Stratagene; and pSVK3, pBPV, pMSG and pSVL available from Pharmacia, and pSGL3 and 4 available from Promega. Other suitable vectors will be readily apparent to the skilled artisan. Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals.

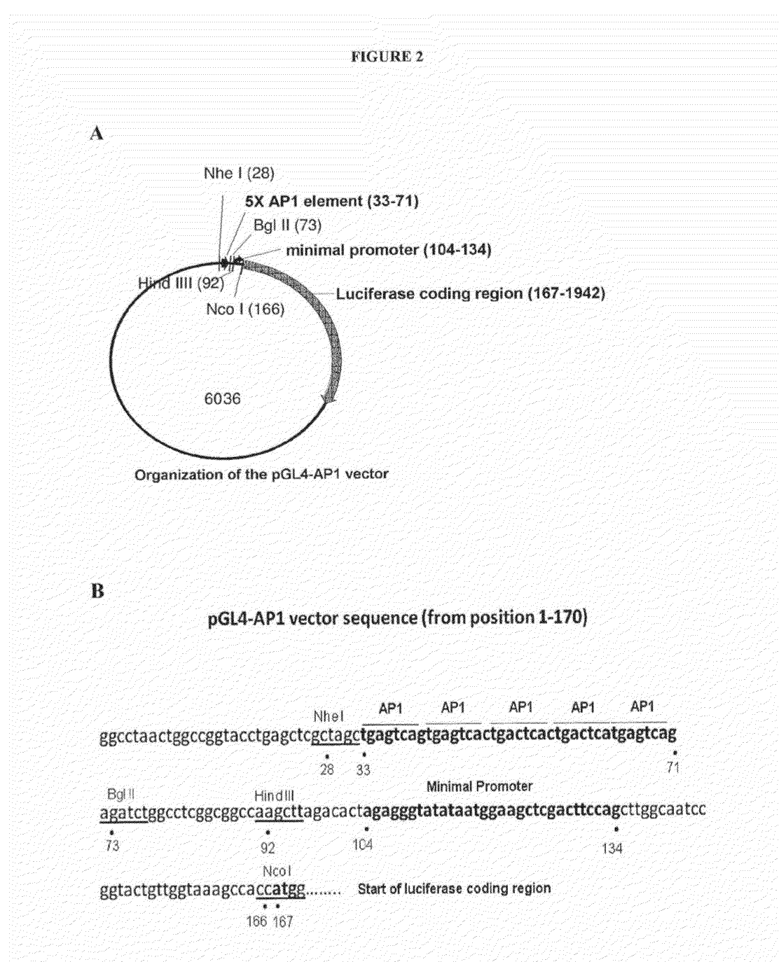

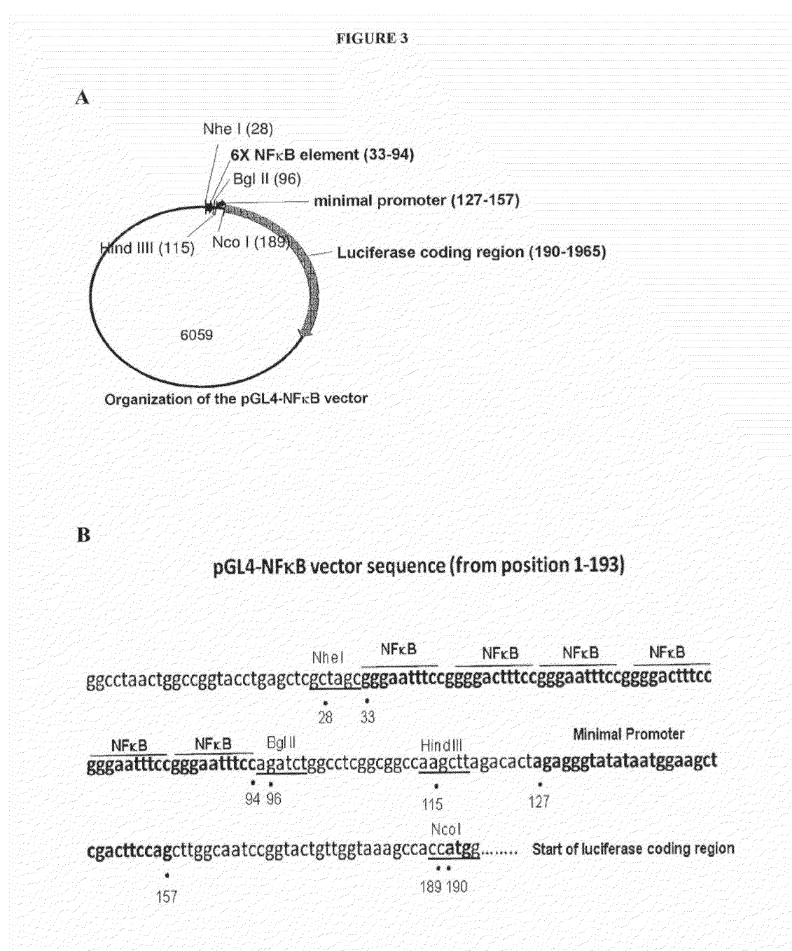

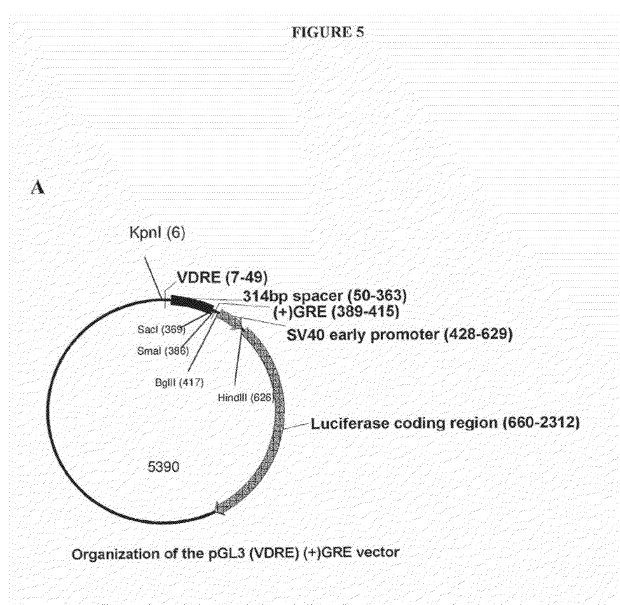

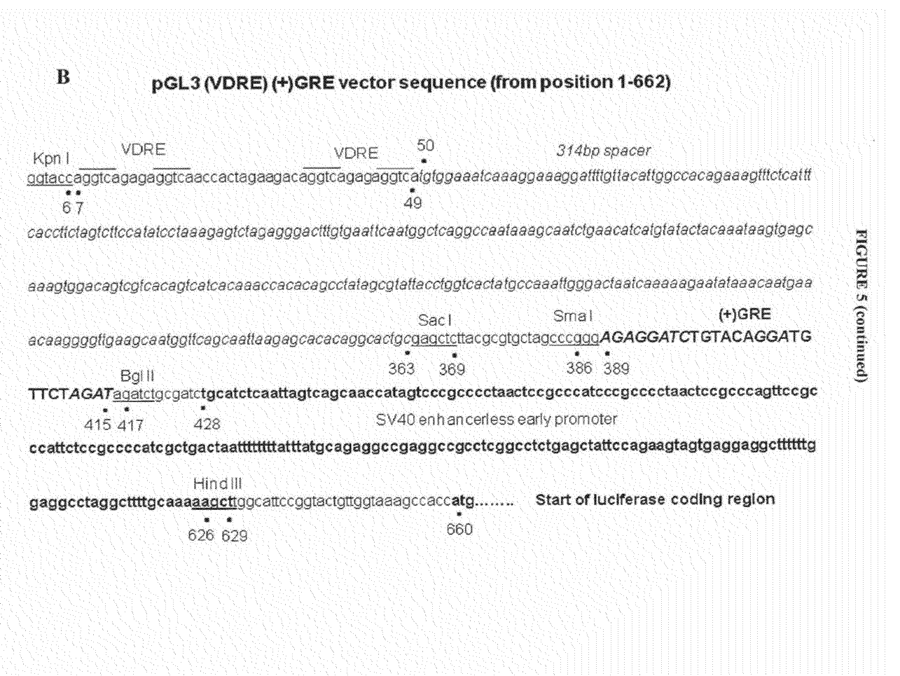

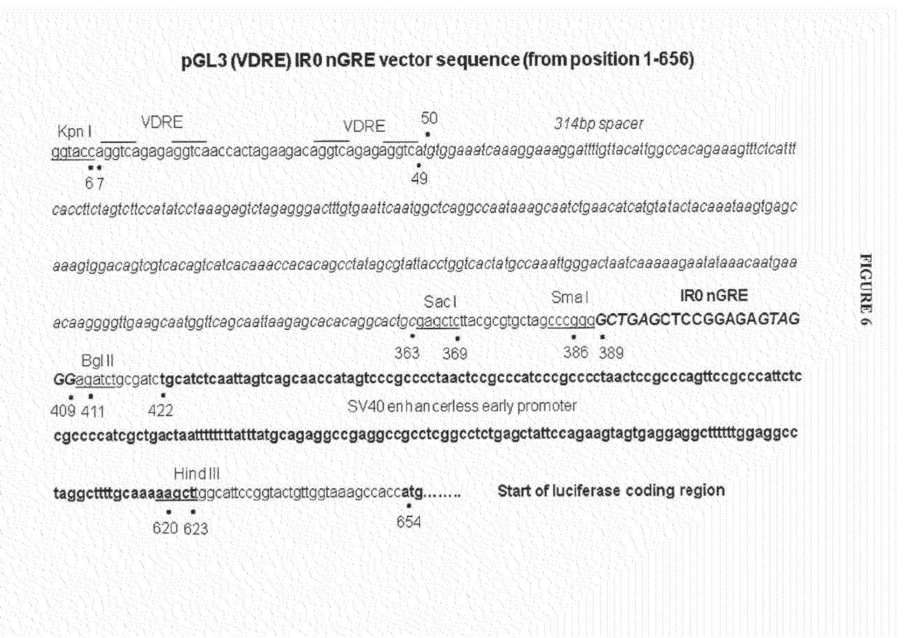

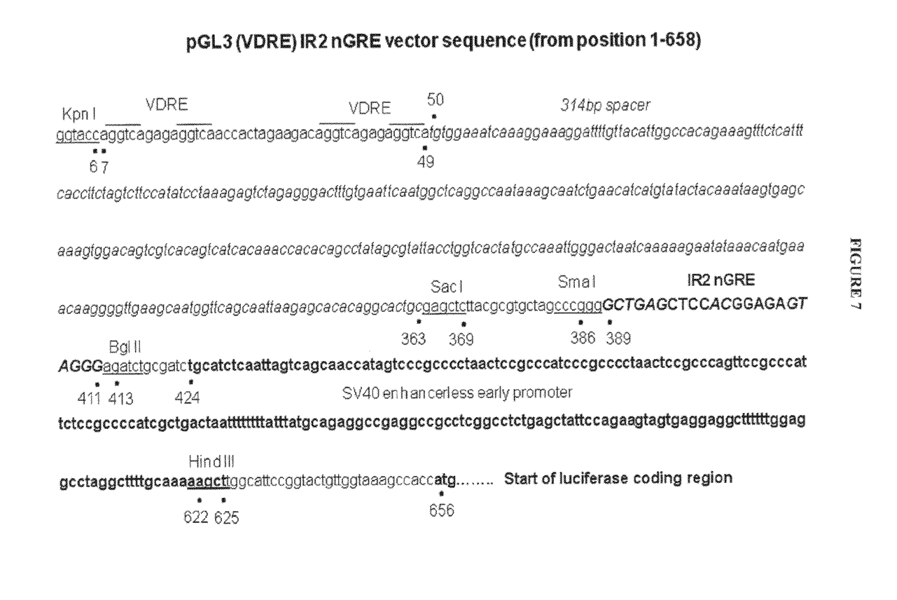

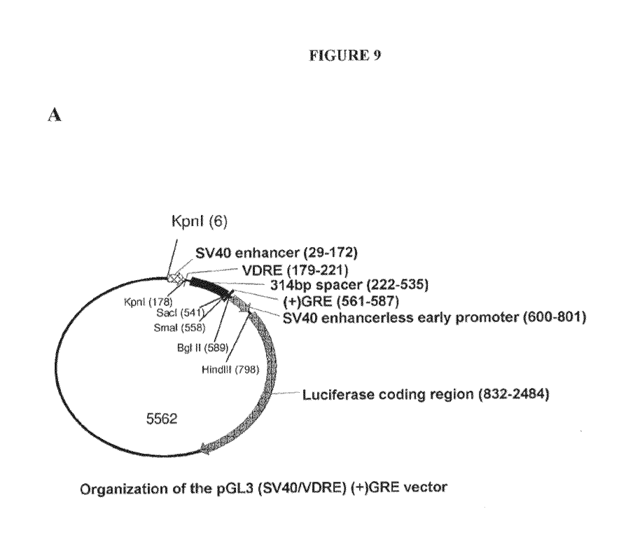

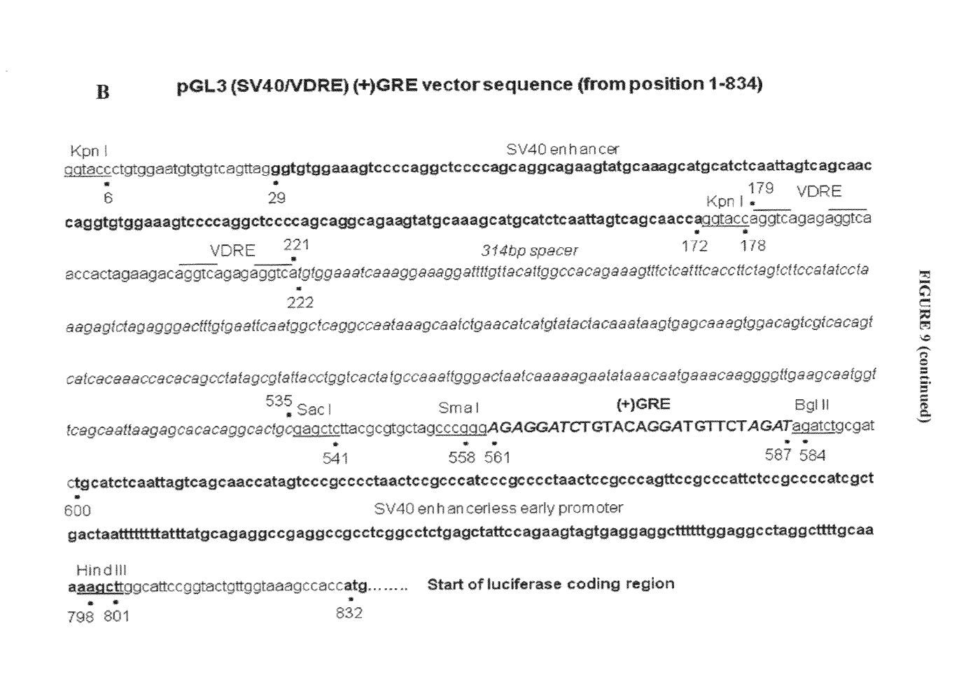

Preferably, in the context of the present invention, and as shown in the experimental section below, said (+) GRE or IR nGRE containing vector is a PGL3 promoter vector plasmid (Promega) containing a SV40 enhancerless early promoter upstream of the luciferase coding region, and containing the SV40 enhancer and/or two copies of consensus VDRE (VDR response element) upstream of the (+) GRE or IR nGRE element (see for example FIGS. 5 and 9). Moreover, said AP1 or NFκB containing vector is preferably a PGL4-NFκB luc plasmid which contains 6 tandem copies of consensus NFκB response element upstream of a SV40 minimal promoter derived from (Promega) PGL4 vector or 6 tandem copies of consensus AP1 response element cloned into said pGL4 vector between the XhoI and BglII restriction sites (see FIGS. 2 and 3). Preferably, said PGL3 and PGL4 vectors are transfected into human A549 cells (CCL-185 ATTC).

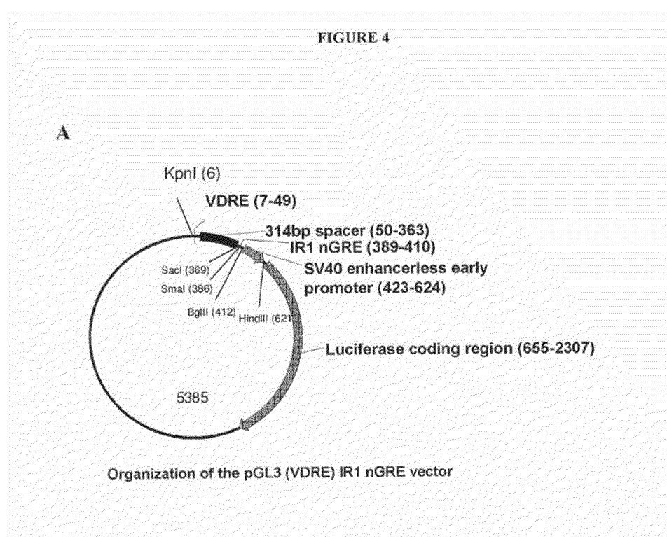

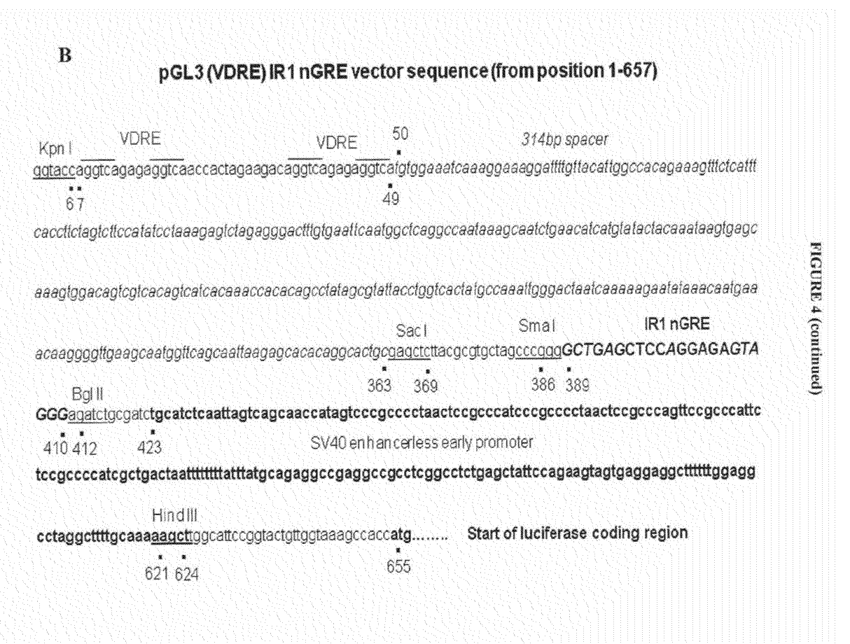

Advantageously, AP1 and NFκB are activated with IL-1β (5 ng/ml) in the case of A549 cells in culture, and by topical application of the phorbolester TPA in the case of skin (1 nmole/cm²), and by LPS (IP, 1 mg/kg) in the case of the liver and intestine. Activation and repression of reporter genes by GC is preferably performed by addition of 1 μM Dept in culture medium. Expression of the reporter genes containing a VDRE (e.g./ vectors in FIGS. 4, 5, 6 and 7) is preferably induced by addition of 1 μM vit D3 in the culture medium.

In a particular embodiment, the measuring step g) comprises the following sub-steps:
g0) measuring the expression level. E0 of each reporter gene in the absence of said candidate molecule and under conditions wherein the expression of said reporter genes of steps b) and c) is significantly repressed by GC, whereas the expression of said reporter gene of step d) is significantly increased by the addition of GC,
g1) measuring the expression level E1 of each reporter gene in the presence of said candidate molecule, all other conditions being the same as in step g0),
and wherein, in said step h), said candidate molecule is selected if:
   E1 is significantly lower than E0 for said reporter gene of step b), and E1 is not significantly lower than E0 for said reporter gene of step c), or
   E1 is significantly lower than E0 for said reporter gene of step b), and E1 is not significantly lower than E0 for said reporter gene of step c), and E1 is not significantly higher than E0 for said reporter gene of step d).

Said reporter gene expression level can be detected by any mean known by the person skilled in the art, for example by quantifying the mRNA level of said gene or by measuring the amount of the reporter-encoded protein as previously described. Preferably, said reporter gene is luciferase, β galactosidase or a fluorescent protein. These assays are well known in the art.

In a preferred embodiment, the first vector which is provided in step b) of the process of the invention has the sequence SEQ ID NO:55 (PGL4-AP1 vector, FIGS. 2A and B) or SEQ ID NO:56 (PGL4-NFFB vector, FIGS. 3A and B).

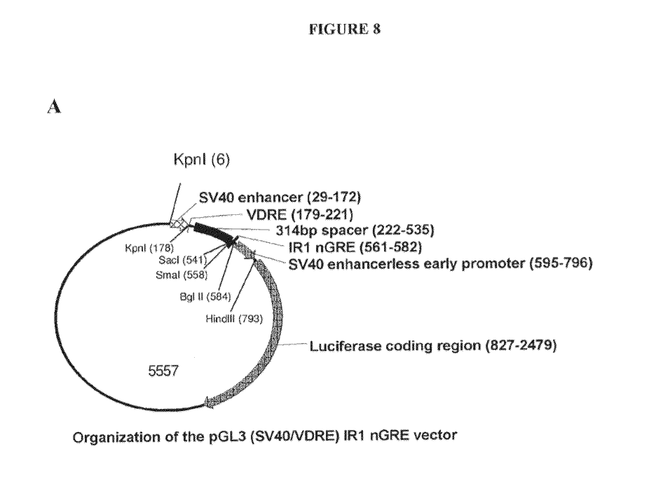

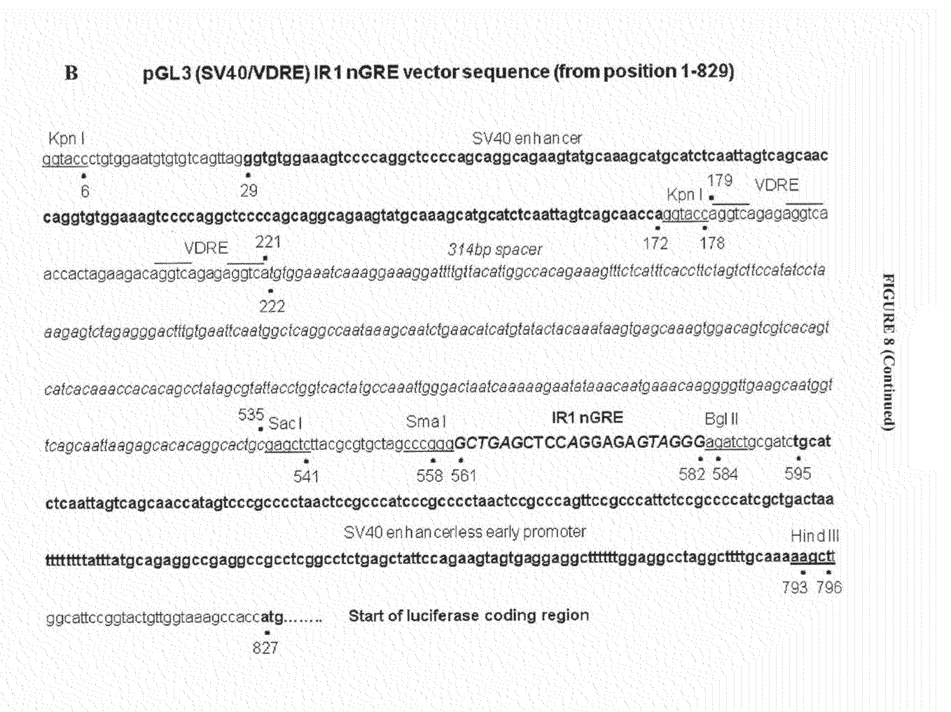

In another preferred embodiment, the second vector which is provided in step c) of the process of the invention has the sequence SEQ ID NO: 49 (PGL3 (SV40/VDRE) IR1 nGRE, FIGS. 8A and B), SEQ ID NO:50 (PGL3(VDRE) IR1 nGRE, FIGS. 4 A and B), SEQ ID NO:52 (PGL3(VDRE) IR2 nGRE, FIG. 7) or SEQ ID NO:51 (PGL3(VDRE) IR0 nGRE, FIG. 6).

In another preferred embodiment, the third vector which is provided in step d) of the process of the invention has the sequence SEQ ID NO:53 (PGL3(SV40/VDRE) (+) GRE, FIGS. 9A and B) or SEQ ID NO:54 (PGL3(VDRE) (+) GRE, FIGS. 5A and B).

As used herein, two gene expression levels will be considered as “similar” or “not significantly lower” or “not significantly higher” if their ratio (i.e. the ratio of their mRNA levels and/or of their protein levels) is comprised between 0.80 and 1.25, preferably between 0.85 and 1.17, preferably between 0.9 and 1.1, and more preferably between 0.95 and 1.05. On the other hand, the expression level of a gene in the presence of said candidate molecule is said to be “significantly lower” than the expression level of the same gene in the absence of said candidate molecule if the expression level of said gene in the presence of said candidate molecule is of maximally 50%, preferably 30%, and more preferably 25% of the expression level of said gene in the absence of said candidate molecule.

In a particular embodiment, the process of the invention comprises the following steps:
a) providing at least one candidate molecule,
b) choosing at least one proinflammatory genomic gene which is known to be transrepressed by GC, and containing among its promoter elements at least one NFκB and/or one AP1 DBS, said gene being significantly expressed in said cells,
c) choosing at least one genomic gene which is known to be transrepressed by GC, and containing among its promoter elements at least one IR nGRE DBS, said gene being significantly expressed in said cells,
d) choosing at least one genomic gene containing among its promoter elements at least one (+) GRE DBS, the expression of said gene being significantly increased by the addition of GC in said cells,
e) selecting conditions wherein NFκB and/or AP1 factors are activated in step b), and selecting conditions wherein said reporter genes of steps b) wherein the expression of said reporter gene of step d) is significantly increased by the addition of GC,
f) measuring the expression level E0 of each gene in said cells in the absence of said candidate molecule, under conditions selected in step e),
g) providing said candidate molecule to said cells and measuring the expression level E1 of each gene in the presence of said candidate molecule, all other conditions being the same as in step f),
h) selecting said candidate molecule if:
   E1 is significantly lower than E0 for said gene of step b), and E1 is not significantly lower than E0 for said gene of step c), or
   E1 is significantly lower than E0 for said gene of step b), and E1 is not significantly lower than E0 for said gene of step c), and E1 is not significantly higher than E0 for said gene of step d).

The present Inventors have found that, when cells are treated with said candidate molecule together with a GR antagonist known to relieve IRnGRE-mediated transrepression (such as RU486), the direct transrepression and transactivation of GC-dependent genes is affected, whereas the indirect transrepression of GC-dependent genes is not significantly affected.

In particular, when cells are treated with said candidate molecule with or without a co-treatment with any GR antagonist known to relieve IRnGRE-mediated transrepression, said candidate molecule is selected only if the expression of a gene which is known to be decreased by GC treatment is decreased by addition of said candidate molecule irrespective of the presence of said antagonist.

In a particular embodiment, the process of the invention therefore preferably further comprises the step of:
providing concomitantly to said candidate molecule the antiglucocorticoid RU486, and
measuring the expression level E2 of one gene chosen in step b) in the presence of said candidate molecule and said antiglucocorticoid, all other conditions being the same as in step f),

and wherein said candidate molecule is selected if E2 is significantly lower than E0 for said gene of step b).

In a preferred embodiment, in step b), said proinflammatory genomic gene which is known to be transrepressed by GC, and which contains among its promoter elements at least one NFκB and/or one AP1 DBS, is chosen in the group consisting of: the COX2 and MMP13, IL4, and IL10 genes.

In another preferred embodiment, in step c), said genomic gene which is known to be transrepressed by GC, and which contains among its promoter elements at least one IR nGRE DBS is chosen in the group consisting of: the BCL3, BHLHB2, ENC1, FGFR3, FOXa2, GEM, MAFK CCND1, CYP26A1, JUND, HSD11β2, PRKCB, K14, RDH11, STRA13, RORα and TNFRSF19 genes.

In another preferred embodiment, in step d), said genomic gene containing among its promoter elements at least one (+) GRE DBS is chosen in the group consisting of: the GILZ, GPX3, GGT1 and ERP27 genes.

Said GC-dependent genes are further identified in Table 2 and Table 3 below:

TABLE 2
NFkB/AP1 DBS containing-, IR nGRE containing-, and (+)GRE
containing genes analyzed in A549 cells

| | Ensembl accession number |
|---|---|
| NFkB/AP1 DBS containing genes | |
| COX2 | ENSG00000073756 |
| MMP13 | ENSG00000137745 |
| IR nGRE containing genes | |
| BCL3 | ENSG00000069399 |
| BHLHB2 | ENSG00000134107 |
| ENC1 | ENSG00000171617 |
| FGFR3 | ENSG00000068078 |
| FOXA2 | ENSG00000125798 |
| GEM | ENSG00000164949 |
| MAFK | ENSG00000198517 |
| (+)GRE containing genes | |
| GILZ | ENSG00000157514 |

TABLE 3
NFkB/AP1 DBS containing-, IR nGRE containing-, and (+)GRE containing
genes analyzed in three mouse tissues

| | Ensembl | IR nGRE | Ensembl | IR nGRE | Ensembl |
|---|---|---|---|---|---|
| | accession | containing | accession | containing | accession |
| Genes | number | genes | number | genes | number |
| NFkB/AP1 | | | | | |
| DBS | | | | | |
| containing | | | | | |
| genes | | | | | |
| COX2 | ENSMUSG00000032487 | COX2 | ENSMUSG00000032487 | COX2 | ENSMUSG00000032487 |
| IL4 | ENSMUSG00000000869 | IL1β | ENSMUSG00000027398 | | |
| IL10 | ENSMUSG00000016529 | | | | |
| IL1β | ENSMUSG00000027398 | | | | |
| TNFα | ENSMUSG00000024401 | | | | |
| MMP13 | ENSMUSG00000050578 | | | | |
| IR nGRE | | | | | |
| containing | | | | | |
| genes | | | | | |
| CCND1 | ENSMUSG00000070348 | CCND1 | ENSMUSG00000070348 | CCND1 | ENSMUSG00000070348 |
| CYP26A1 | ENSMUSG00000024987 | JUND | ENSMUSG00000071076 | JUND | ENSMUSG00000071076 |
| HSD11b2 | ENSMUSG00000031891 | PRKCB | ENSMUSG00000052889 | PRKCB | ENSMUSG00000052889 |
| K14 | ENSMUSG00000045545 | RDH11 | ENSMUSG00000066441 | RDH11 | ENSMUSG00000066441 |
| PRKCB | ENSMUSG00000052889 | RORα | ENSMUSG00000032238 | RORα | ENSMUSG00000032238 |
| STRAL3 | ENSMUSG00000030103 | STRAL3 | ENSMUSG00000030103 | STRAL3 | ENSMUSG00000030103 |
| TNFRSF19 | ENSMUSG00000060548 | TNFRSF19 | ENSMUSG00000060548 | TNFRSF19 | ENSMUSG00000060548 |
| (+)GRE | | | | | |
| containing | | | | | |
| genes | | | | | |
| GPX3 | ENSMUSG00000018339 | GPX3 | ENSMUSG00000018339 | GPX3 | ENSMUSG00000018339 |
| | | | | GGT1 | ENSMUSG00000006345 |
| | | | | ERP27 | ENSMUSG00000030219 |

In a preferred embodiment, GR-expressing cells are in vitro cultured cells, preferably human lung epithelial carcinoma cells A549, or cells from an animal tissue. More preferably, said animal is a mouse and said tissue is epidermis, liver or intestinal epithelium.

When GR-expressing cells are human lung epithelial carcinoma cells A549, said gene of step b) is preferably chosen from the COX2 or MMP13 genes, said gene of step c) is preferably chosen from the group consisting of: BCL3, BHLHB2, ENC1, FGFR3, FOXa2, GEM and MAFK genes, and said gene of step d) is preferably the GILZ gene.

When GR-expressing cells are from a tissue of an animal, said gene of step b) is preferably chosen in the group consisting of: the COX2, IL4, IL1β, TNFα, MMP13 and IL10 genes, said gene of step c) is preferably chosen in the group consisting of: CCND1, CYP26A1, JUND, HSD11β2, PRKCB, K14, RDH11, STRA13, RORα and TNFRSF19 genes and said gene of step d) is preferably chosen in the group consisting of the GPX3, GGT1 and ERP27 genes.

All these genes have been defined in Tables 2 and 3 above.

In a particular embodiment, said animal tissue is epidermis and the process of the invention comprises the following steps:
a) providing a candidate molecule and laboratory animals,
b) measuring, in said laboratory animals, after topical treatment of skin with the phorbol ester TPA (for example: 1 nmole/cm²) in order to activate NFκB and/or AP1, the expression level E0 of a gene belonging to the group of inflammatory genes containing at least one NFκB and/or one AP1 DBS, said gene being preferably chosen in the group consisting of: the COX2, IL4, IL1β, TNFα, MMP13 and IL10 genes,
c) measuring the expression level E0 of a gene containing a IR nGRE DBS, said gene being preferably chosen in the group consisting of: the CCND1, CYP26A1, HSD11β2, PRKCB, K14, STRA13, and TNFRSF19 genes,
d) measuring the expression level E0 of a gene containing a (+) GRE DBS, said gene being preferably the GPX3 gene,
e) topically treating the skin of said laboratory animals with said candidate molecule,
f) measuring the expression level E1 of each gene of steps b), c) and d) in the presence of said candidate molecule,
g) selecting said candidate molecule if:
   E1 is significantly lower than E0 for said gene of step b), and E1 is not significantly lower than E0 for said gene of step c), or
   E1 is significantly lower than E0 for said gene of step b), and E1 is not significantly lower than E0 for said gene of step c), and E1 is not significantly higher than E0 for said gene of step d).

In a particular embodiment, said animal tissue is liver and the process of the invention comprises the following steps:
a) providing a candidate molecule and laboratory animals,
b) measuring, in the liver tissue of said laboratory animals, after intraperitoneal treatment of lipopolysaccharide (LPS) (for example: IP, 1 mg/kg) in order to activate NFκB and/or AP1, the expression level E0 of a gene belonging to the group of inflammatory genes containing at least one NFκB and/or one AP1 DBS, said gene being preferably the COX2 gene,
c) measuring in the same tissue as in b) the expression level E0 of a gene containing a IR nGRE DBS, said gene being preferably chosen in the group consisting of: the CCND1, JUND, PRKCB, RDH11, RORα, STRA13 and TNTFRSF19 genes,
d) measuring in the same tissue as in b) the expression level E0 of a gene containing a (+) GRE DBS, said gene being preferably chosen in the group consisting of: the GPX3, GGT1 and ERP27 genes,
e) administering said candidate molecule intraperitoneally in said laboratory animals,
f) measuring in the same tissue as in b) the expression level E1 of each gene of steps b), c) and d) in the presence of said candidate molecule,
g) selecting said candidate molecule if:
   E1 is significantly lower than E0 for said gene of step b), and E1 is not significantly lower than E0 for said gene of step c), or
   E1 is significantly lower than E0 for said gene of step b), and E1 is not significantly lower than E0 for said gene of step c), and E1 is not significantly higher than E0 for said gene of step d).

In another particular embodiment, said animal tissue is intestinal epithelium and the process of the invention comprises the following steps:
a) providing a candidate molecule and laboratory animals,
b) measuring, in the intestinal epithelium tissue of said laboratory animals, after intraperitoneal treatment of lipopolysaccharide (LPS) (for example: 1 mg/kg) in order to activate NFκB and/or AP1, the expression level E0 of a gene belonging to the group of inflammatory genes containing at least one NFκB and/or one AP1 DBS, said gene being preferably chosen in the group consisting of: the COX2 and the IL1β genes,
c) measuring in the same tissue as in b) the expression level E0 of a gene containing a IR nGRE DBS, said gene being preferably chosen in the group consisting of: the CCND1, JUND, PRKCB, RDH11, STRA13, RORα and TNTFRSF19 genes,
d) measuring in the same tissue as in b) the expression level E0 of a gene containing a (+) GRE DBS, said gene being preferably the GPX3 gene,
e) administering said candidate molecule intraperitoneally in said laboratory animals,
f) measuring in the same tissue as in b) the expression level E1 of each gene of steps b), c) and d) in the presence of said candidate molecule,
g) selecting said candidate molecule if:
   E1 is significantly lower than E0 for said gene of step b), and E1 is not significantly lower than E0 for said gene of step c), or
   E1 is significantly lower than E0 for said gene of step b), and E1 is not significantly lower than E0 for said gene of step c), and E1 is not significantly higher than E0 for said gene of step d).

Said laboratory animals are preferably chosen from mice, rats, and rabbits, and are more preferably mice.

In another embodiment, the process of the invention comprises the steps of:
a) providing a candidate molecule and laboratory animals,
b) measuring, in one tissue of said laboratory animals, after topical or intraperitoneal treatment of lipopolysaccharide (LPS) or TPA in order to activate NFκB and/or AP1, the expression level E0 of at least one pro-inflammatory gene containing at least one NFκB and/or one AP1 DBS,
c) administering said candidate molecule topically or intraperitoneally in said laboratory animals,
d) measuring in the same tissue as in b) the expression level E1 of said gene in the presence of said candidate molecule,
e) administering topically or intraperitoneally in said laboratory animals, concomitantly to said candidate molecule, a GR antagonist having an IR nGRE mediated transrepression activity similar to that of the antiglucocorticoid RU486,
f) measuring in the same tissue as in b) the expression level E2 of said gene in the presence of said candidate molecule and said GR antagonist,
g) selecting said candidate molecule if E2 is significantly lower than E0 for said gene.

Said pro-inflammatory gene is chosen from: the COX2, IL4, IL1β, TNFα, MMP13 and IL10 genes if said tissue is epidermis, from the COX2 and the IL1β genes, if said tissue is intestinal epithelium and is COX2 gene if the tissue is liver.

The administration of said candidate molecule and/or GR antagonist in steps c) and/or e) is performed either by topical application (for example for regulating the genes expressed in epidermis) or by injection (intraperitoneally or intravenously), said administration being thus not a chirurgical act for said laboratory animal.

Preferably, said “GR antagonist having the same IR nGRE mediated transrepression activity as the antiglucocorticoid RU486” designates RU486 itself.

It is reminded that the expression level of a gene in the presence of said candidate molecule is said to be “significantly lower” than the expression level of the same gene in the absence of said candidate molecule if the expression level of said gene in the presence of said candidate molecule is of maximally 50%, preferably 30%, and more preferably 25% of the expression level of said gene in the absence of said candidate molecule.

In other words, in the process of the invention, said GR-expressing cells are:

a) epidermal cells of the skin of an animal, preferably a mouse, on which said candidate molecule has been administered topically without or with the antiglucocorticoid RU486, wherein said gene in step b) is chosen in the group consisting in: the COX2, HA, IL4, IL1β, TNFα, MMP13 and IL10 genes, said gene of step c) is chosen in the group consisting in: the CCND1, CYP26A1, HSD11β2, PRKCB, K14. STRA13, and TNFRSF19, and said gene of step d) is the GPX3 gene,

b) cells obtained from the liver of an animal, preferably a mouse, on which said candidate molecule has been administered without or with the antiglucocorticoid RU486, wherein said gene in step b) is the COX2 gene, said gene of step c) is chosen in the group consisting in: the CCND1, JUND, PRKCB, RDH11, STRA13, RORα and TNFRSF19 genes, and said gene of step d) is chosen in the group consisting of: the GPX3, GGT1 and ERP27 genes, or

c) cells obtained from the intestinal epithelium of an animal, preferably a mouse, on which said candidate molecule has been administered without or with the antiglucocorticoid RU486, wherein said gene in step b) is chosen in the group consisting in: the COX2 and IL1β genes, said gene of step c) is chosen in the group consisting in: the CCND1, JUND, PRKCB, RDH11, STRA13, RORα and TNFRSF19 genes, and said gene of step d) is the GPX3 gene.

In a second aspect, the present invention is drawn to the use of an IR nGRE DBS present in recombinant vectors and animal genomes for identifying a molecule exhibiting anti-inflammatory activities of GCs and having reduced GC-dependent IR nGRE-mediated direct transrepression activity.

Preferably, said IR nGRE DBS is chosen in the group consisting of: sequence SEQ ID NO:1 (IR1 nGRE), SEQ NO:2 (IR0 nGRE), SEQ ID NO:3 (IR2 nGRE), or tolerable variants thereof.

More preferably, said tolerable variant of SEQ ID NO:1 (IR1 nGRE) being preferably chosen in the group consisting of SEQ ID NO:11 to 27, and said tolerable variant of SEQ ID NO:3 (IR2 nGRE) being preferably chosen in the group consisting of: SEQ ID NO:28 to 48.

In a third aspect, the present invention is drawn to the use of the antiglucocorticoid RU486 or of any GR antagonist having an IR nGRE-mediated transrepression activity similar to that of RU486, for identifying a molecule exhibiting anti-inflammatory activities of GCs and having reduced GC-dependent IR nGRE-mediated direct transrepression activity.

In a fourth aspect, the present invention is drawn to an isolated IR nGRE DNA binding site, chosen in the group consisting of: sequence SEQ ID NO:11 (IR1 nGRE), SEQ ID NO:2 (IR0 nGRE), SEQ ID NO:3 (IR2 nGRE), or tolerable variants thereof.

In a fifth aspect, the present invention is drawn to an isolated vector containing an IR nGRE DBS as defined above. Preferably, said vector is chosen in the group consisting of: SEQ ID NC): 49 (PGL3 (SV40/VDRE) IR1 nGRE), SEQ ID NO:50 (PGL3 (VDRE) IR1 nGRE), SEQ ID NO:51 (PGL3(VDRE) IR0 nGRE), and SEQ ID NO:52 (PGL3(VDRE) IR2 nGRE).

By “isolated” vector is intended a nucleic acid molecule which has been removed from its native environment. Examples of isolated DNA molecules include recombinant DNA molecules maintained in heterologous host cells or purified (partially or substantially) DNA molecules in solution.

This isolated vector can be advantageously used in the screening process of the invention or in any other process intended to select molecules exhibiting anti-inflammatory of GCs, with reduced GC-dependent IR nGRE-mediated direct transrepression activity.

### EXAMPLES

### I. Material and Methods

Mice. For topical treatment, 1 nmole (nm)/cm² MC903, at-RA or TPA; 6 nm/cm² FA, Dex or RU24858; and 90 nm/cm² RU486 were used. For systemic use, 100 ng/kg body weight active Vit D3, 8 mg/kg Dex and 64 mg/kg RU486 was intraperitoneally injected. GRdim mice were from the European Mouse Mutant Archives (EM:02123). Breeding, maintenance and experimental manipulation of mice were approved by the Animal Care and Use Committee of the IGBMC.

Cell culture experiments. A549 human lung epithelial cells (CCL-185, ATCC) were maintained in DMEM/HAM F 12 (1:1) medium containing 10% foetal calf serum (FCS) and gentamycin. MLE12 mouse lung epithelial cells (CRL-2110, ATCC) were maintained in DMEM/Ham-F12 (1:1) medium containing 2% FCS, 5 ug/ml insulin, 10 ug/ml apo-trans bovine, 35 nM sodium selenite, 10 nM β estradiol, 10 mM HEPES and gentamycin. Cells were transfected using Fugene 6 reagent, as instructed (Roche). For RNA isolation from A549 cells, cells were seeded at 50% density in 60 mm plates. 24 hours post-seeding, complete medium was replaced with medium containing charcoal-treated serum. 24 hours later, 1 μM active vitamin D3 [1α, 25 (OH)2 Vitamin D3,], 300 nM FA, 4.5 μM RU486 (final concentration) or vehicle (acetone) were added to the medium for 6 hours. For ChIP assay, cells were treated with indicated compounds for 2 hours, followed by cross-linking by addition of formaldehyde (1% final concentration) to the medium and incubation on a rotary shaker for 10 minutes at room temperature. Cross-linking was stopped by adding 2M glycine (0.125M final concentration) and incubation for 5 minutes under similar condition, followed by 2× washing in ice-cold PBS. Cells were scraped using a rubber policeman, pelleted (400 g, 5 minutes) at 4° C., washed once in ice-cold PBS, snap frozen in liquid nitrogen and stored at −70° C. before proceeding for ChIP assay.

For ChIP assays to test GR and cofactor binding to the nGRE, +GRE or NFκB region of various luciferase reporter plasmids, ˜60% confluent A549 cells in 100 mm TC dish were transfected with 2 μg of respective plasmids using fugene 6 reagent (Roche) and maintained in charcoal treated FCS containing medium. 44 hours post-transfection, 500 nM FA or Dex or RU24858 and/or 5 μM RU486, 5 ng/ml IL-1β (final conc.) was added to the medium for 2 hours, followed by formaldehyde treatment and processing as described above.

Luciferase reporter assay. A549 cells seeded on 24-well tissue culture plates overnight at 70% confluency were transfected with 100 ng pCMV β galactosidase, 200 ng pGL3 reporter plasmids and wherever indicated, with 600 ng pSG5 hVDR plasmid (Green et al., 1988) into each well and maintained in medium containing charcoal treated FCS. 24 hours post-transfection, medium was changed and different compounds were added to it for 6 hours. Final concentrations of the compounds are: FA, 500 nM; Dex or RU24858, 300 nM or 500 nM (wherever 300 nM Dex or RU was used, it is indicated in the figure, in all other cases, 500 nM concentration was used); active vitamin D3, 1 μM; IL-1β, 5 ng/ml; BAY 11-7082, 5 μM, JNK inhibitor II, 25 μM. Luciferase assay was carried out as instructed (Promega). Normalized values are reported as the mean±SEM; each value originates from at least three individual transfections with assays performed in duplicate.

ChIP assay. Isolated epidermis and intestinal epithelial cells were cross-linked in 1% formaldehyde followed by ChIP assay, as reported (Vaisanen et al., 2005).

Nuclear run-on, EMSA, 3C and Luciferase assays. Nuclear Run-on and EMSA (Carey and Smale., 2001), 3C (Liu et al., 2005) and Luciferase assays (Promega kit) were as described.

Real time PCR. Total RNA was reverse transcribed using hexamers, followed by Q-PCR, as reported (Li et al., 2005).

Statistics. Data are represented as mean±SEM of at least three independent experiments, and were analyzed using sigmastat (Systat Software) by the Student t test. P<0.05 was considered significant.

Bioinformatics analysis. hg19 (human) and mm9 (mouse) repeat masked genome assembly was used to identify genome wide distribution of IR nGRE motifs. Gene functional annotation was performed using DAVID programme.

### II. Results

### A) Glucocorticoid-Induced GR-Mediated Transcriptional Repression of TSLP Expression Involves a Negative GRE Located in the TSLP Promoter Region.

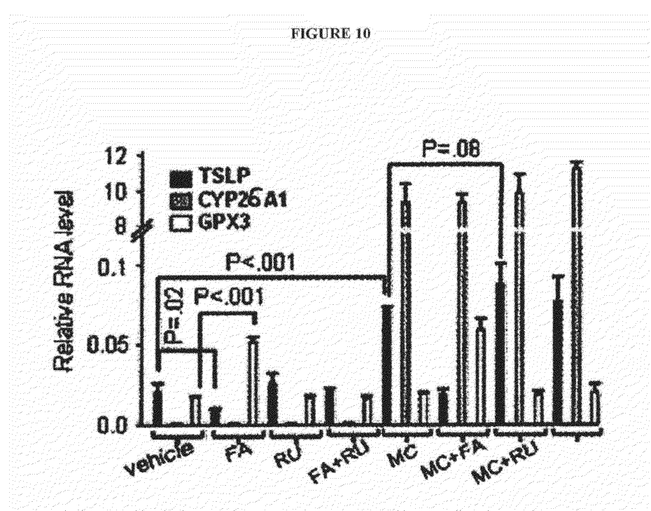

The GC agonist fluocinolone acetonide (FA) was applied to ears of mice concomitantly treated with the “low-calcemic” Vitamin D3 (VitD3) analog MC903 (Calcipotriol; hereafter called MC) to trigger TSLP expression (Li et al., 2006). In wild type (WT) mice, FA application inhibited basal TSLP RNA level by ˜50%, which interestingly could be relieved by co-application of the GC antagonist RU486 (mifepristone, hereafter named RU), while MC-induced increase of TSLP RNA, which was fully blocked by FA, was also restored by RU co-treatment. As expected, the expression of the GC-inducible GPX3 (glutathione peroxidase 3) gene which harbours a (+)GRE (Tuckermann et al., 1999) was enhanced by FA (FIG. 10) and inhibited by RU co-treatment, while FA or RU had no effect on MC-dependent expression of the CYP24A1 gene (a VitD3 target) (FIG. 10).

The GR involvement in FA-induced inhibition of MG-induced TSLP expression in keratinocytes was demonstrated using adult mice in which GR was selectively ablated in keratinocytes (GR^{ep-/—} mice). Although the basal TSLP level was similar in vehicle-treated WT and GR^{ep-/-} mice, FA blocked MC-induced TSLP expression in WT, but not in GR^{ep-/-} mice (not shown). MC treatment was more efficient in GR^{ep-/-} than in WT mice, indicating that endogenous GCs may partially inhibit MC-induced TSLP expression in WT epidermis. Nuclear run-on assays demonstrated that GR-mediated FA inhibition of TSLP expression was transcriptional (not shown),

As neither NFκB nor AP1 are involved in TSLP induction by MC in epidermis (unpublished data), its repression was unlikely to be mediated by a “tethering” GRE. A bioinformatics analysis of 20 kb of DNA located upstream and downstream from the mouse and human TSLP translation start-site (+1) did not reveal any (+)GRE or known “composite” activating or repressing GRE, but unveiled the presence of a palindromic sequence consisting of two inverted repeated (IR) motifs separated by one by (called hereafter IR1 nGRE), in the upstream promoter region of both mouse (m) and human (h) TSLP genes. Recombinant human GR protein in electrophoretic mobility shift (EMSA) and supershift assays with GR antibody showed that this putative mTSLP IR1 nGRE and its human counterpart, as well as the TAT (+)GRE (Meijsing et al., 2009), bound to the GR protein. These bindings were specific, as shown by lack of GR binding to a mutant (+)GRE and to three mTSLP IR1 nGRE mutants. Complexes formed between the recombinant GR and either putative IR1 nGREs or (+)GRE similarly migrated (data not shown).

B) the Repressing Activity of the TSLP nGRE Tolerates Changes in Spacing and/or Sequence of its Repeated Motifs.

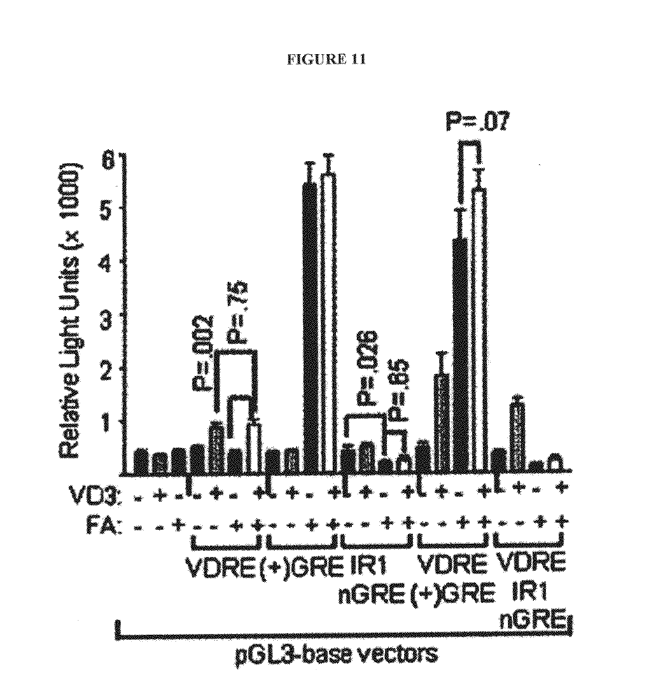

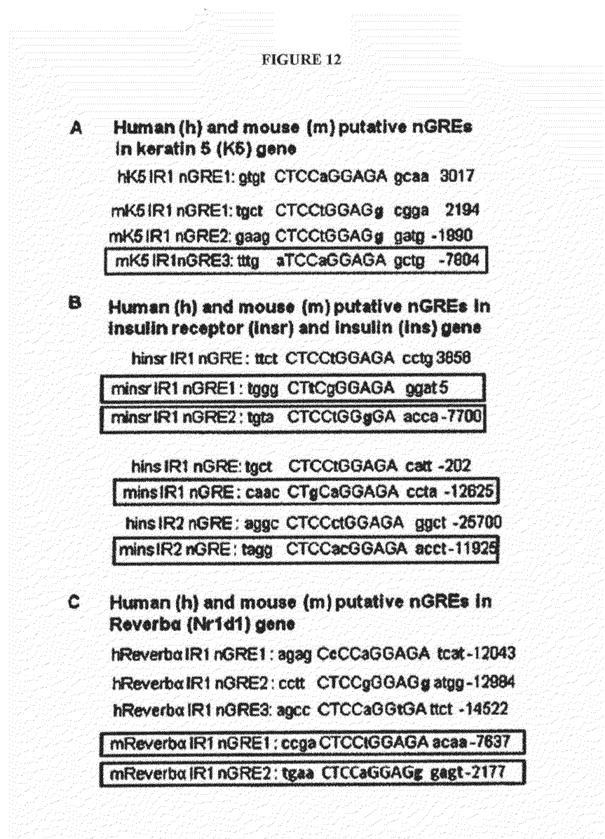

To investigate whether additional DNA elements could be required to generate a repressing activity, the TSLP IR1 nGRE was inserted upstream of an enhancerless SV40 early promoter located 5′ to the luciferase coding sequence of pGL3 vector, A VDRE separated from the IR1 nGRE by a 314 bp-long DNA segment devoid of any known transregulator binding site (not shown) was inserted to generate a luciferase-expressing reporter plasmid (pGL3(VDRE) IR1 nGRE, FIG. 4), which was transfected into A549 cells, followed by addition of VitD3 and/or FA. FA addition did not affect luciferase expression in absence of IR1 nGRE, whereas its presence resulted in decreased expression (which could be prevented by RU addition) of basal and VDRE-mediated VitD3-induced transcription (FIG. 11). As expected, FA-induced increase in luciferase expression was observed when IR1 nGRE was replaced by a (+)GRE (PGL3(VDRE) (+)GRE vector, FIG. 5). The TSLP IR1 nGRE exhibited a similar FA-inducible repressing activity when embedded in 413 bp of its natural environment (not shown). Thus, on its own, TSLP IR1 nGRE is sufficient to mediate a FA-inducible repressing activity. Replacing, in the reporter, IR1 nGRE by a mutant to which recombinant GR does not bind resulted in the loss of GR binding to the IR nGRE region, and in no FA-inducible repressing activity (data not shown), thus supporting the conclusion that GR binds directly to TSLP nGRE in cultured cells.

To investigate whether the 1 bp spacer between the inverted repeated motifs of TSLP IR1 nGRE was critical for its repressing function, pGL3-based luciferase plasmids containing the VDRE and TSLP nGRE motifs spaced by 0 to 5 bases (IR0 to IR5 nGREs) were transfected into A549 cells. ChIP assays showed that, upon FA addition, GR similarly bound IR1 and IR2 nGREs, whereas its binding was less efficient on IR0 nGRE, and not detectable on IR3, IR4 and IR5 nGREs (not shown). Accordingly, upon FA addition, the decrease in basal and VitD3-induced luciferase activity was stronger for IR1 and IR2 than for IR0 nGRE, whereas no significant change could be detected for IR3, IR4 and IR5 nGREs (not shown).

pGL3-based luciferase plasmids containing VDRE and TSLP IR1 nGREs (FIG. 4A), in which individual bases had been changed one by one, were used to study whether non-canonical IR1 nGREs could function as efficient nGREs. With one exception, at least a one base pair change was “tolerable” at any position of the TSLP IR1 nGRE and did not impair its activity in vitro, suggesting that GR bound to “degenerate” IR1 elements might also mediate GC-induced direct repression in vivo (see below). A similar analysis carried out with plasmids containing IR2 nGREs showed that a one base pair change was “tolerable” at any position of IR2 nGRE (not shown). The IR1 nGRE having efficient GR binding are SEQ ID NO: 11 to SEQ ID NO:27, and the IR2 nGRE having efficient GR binding are SEQ ID NO:28 to SEQ ID NO:48.

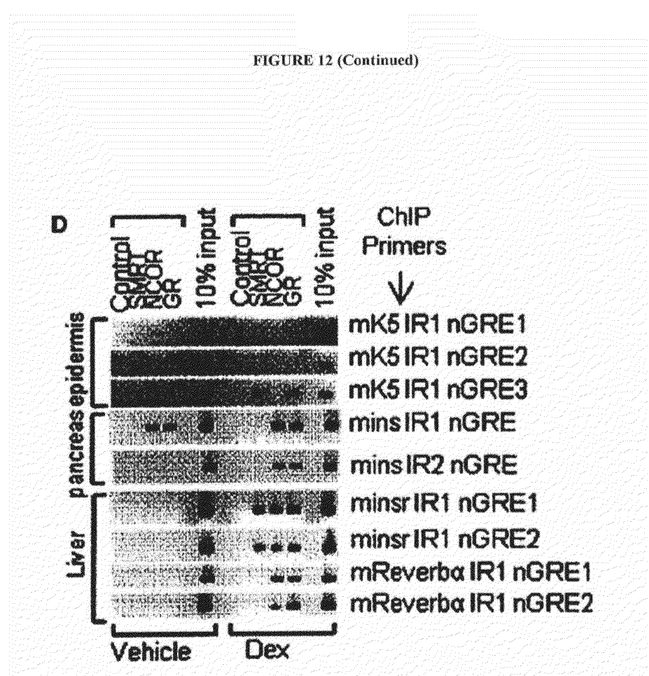

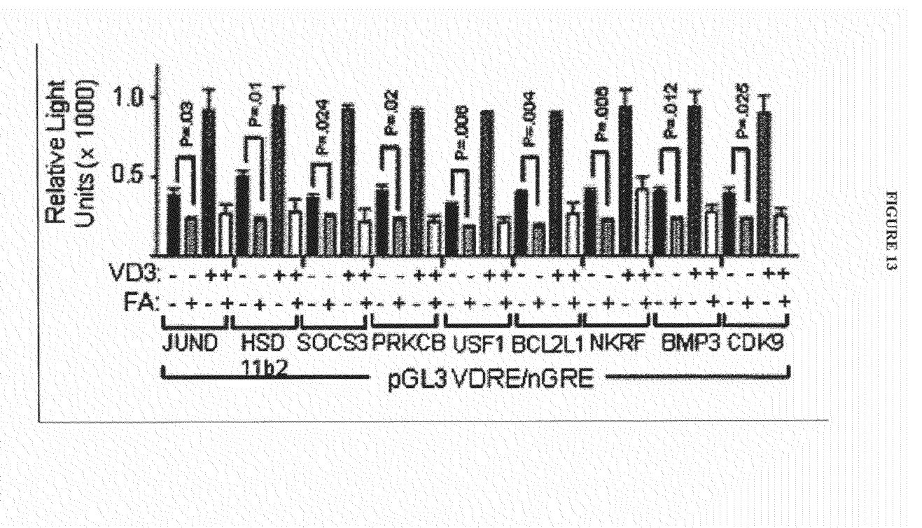

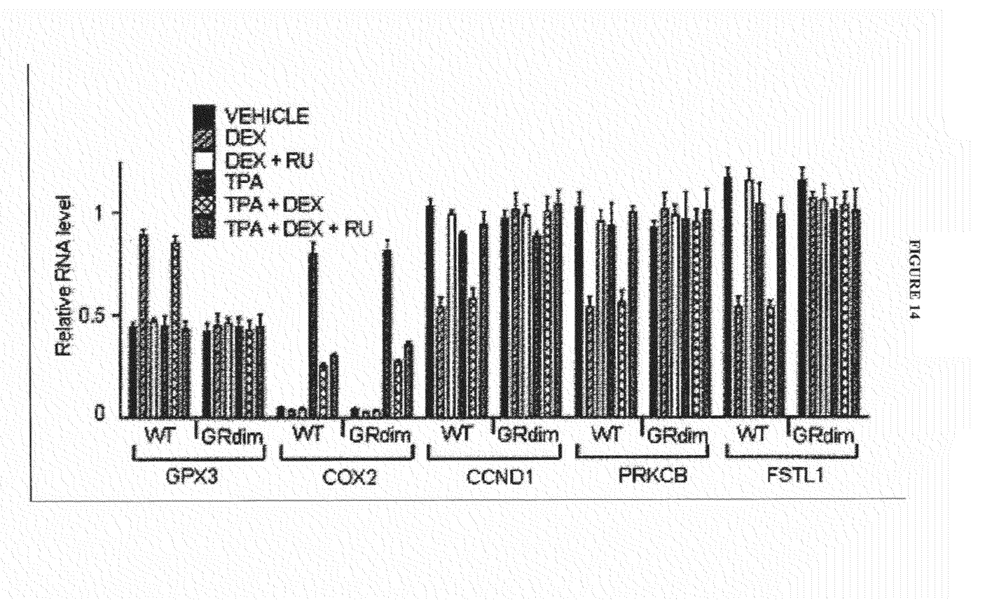

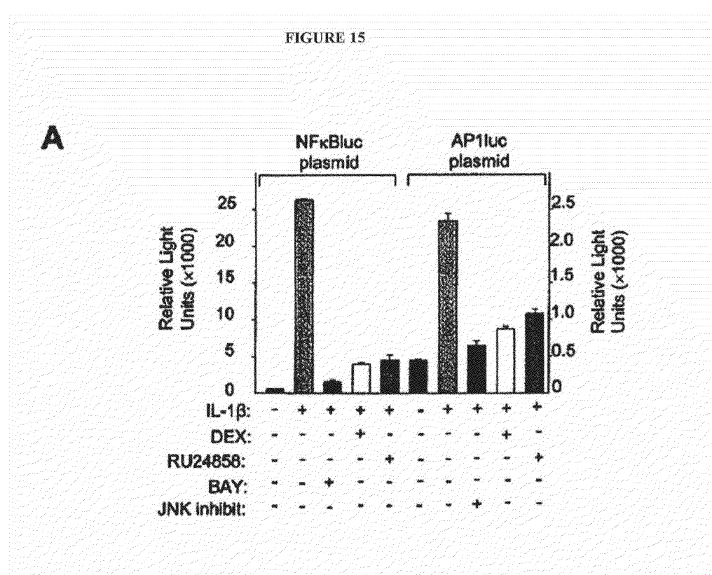

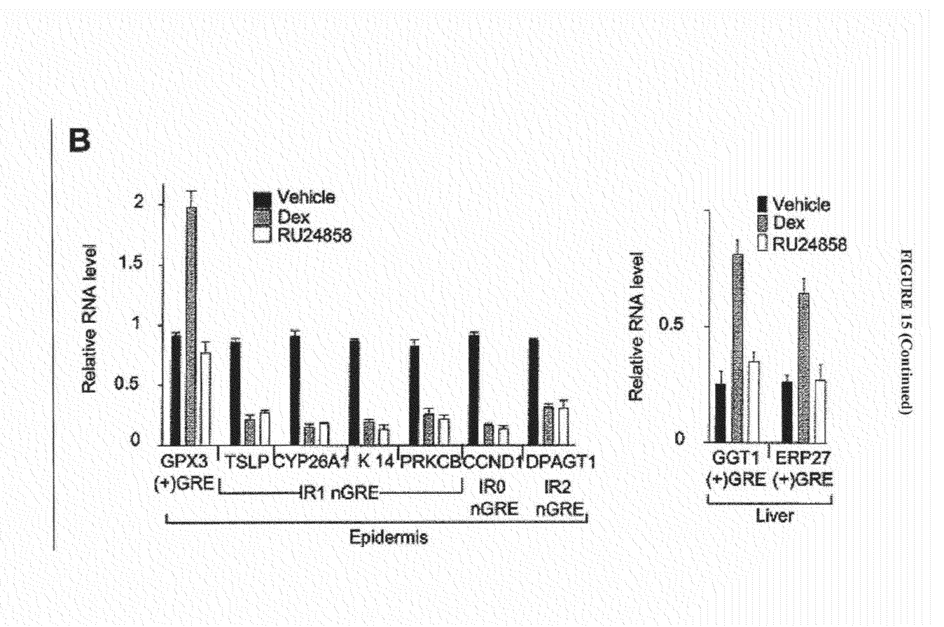

As Keratin 5 (K5), known to be down-regulated by GCs (Ramot et al., 2009), was not present in the list of human/mouse orthologues that contain a canonical nGRE, it was studied whether this absence could reflect the presence of a canonical IR1 nGRE in human K5, while a “tolerable” change would exist in its mouse orthologue, or vice-versa. One canonical IR1 nGRE in human K5 gene and 3 putative nGREs in its mouse orthologue were found, each of them exhibiting one “tolerable” change in vitro (FIG. 12 A). Only one of them (mK5 IR1 nGRE3, FIG. 12 D) allowed formation of a GR-SMRT repressing complex in epidermis of Dex-treated mice, suggesting that not all in vitro “tolerable” IR1 nGRE variants are functional in a given tissue.

Similarly, the insulin (ins) and insulin receptor (insr) genes were studied, as their expression was reported to be down-regulated by GCs (Delaunay et al., 1997). A canonical IR1 nGRE was found in the human insulin receptor gene (hinsr IR1 nGRE), and two IR1 nGREs, each bearing one “tolerable” change, were present in the mouse (FIG. 12B). Upon Dex treatment, functional GR-SMRT/NCoR repressing complexes were assembled on both mouse insulin receptor IR1 nGRE variants (minsr IR1 nGRE1 and 2 in FIG. 12D). Remarkably, in addition to a canonical IR2 nGRE in both human and mouse insulin genes, a “tolerable” IR1 nGRE variant was present in mouse insulin gene, while a canonical IR1 nGRE was present in the human gene (FIG. 12 B).

It was also looked at Reverbα, as its down-regulation by GCs was previously reported (Duez and Staels, 2008, and Refs therein). A canonical IR1 nGRE was found in mouse, but not in human Reverbα gene, which contains 3 IR1 nGREs each bearing one “tolerable” variant (FIG. 12C), Note that, in the mouse, this gene also contains an IR1 nGRE variant (mReverbα IR1 nGRE2), that is identical to one of the human nGRE variants, and appears to be as functional as the mouse canonical IR1 nGRE1, as judged from ChIP assays with liver extracts (FIG. 12D).

C) Mouse Genes that Contain IR0, IR1 and IR2 nGREs Conserved in their Human Orthologues are Repressed Upon GC Agonist Treatment In Vivo.

Bioinformatics analyses of mouse and human genomes revealed thousands of genes containing IR elements made up of inverted repeated motifs identical to those of TSLP (CTCC and GGAGA) with either no (IR0), 1 (IR1) or 2 (IR2) bp spacers. A comparison of mouse and human orthologues indicated conservations of such IR elements (51 IR0, 379 IR1 and 566 IR2). Within these orthologue gene families, 35 IR0, 50 IR1 and 50 IR2 nGRE-containing mouse genes (Table 4) were randomly chosen to investigate whether (i) they were expressed in epidermis, intestinal epithelium and liver, (ii) their expression was inhibited by the GC agonist Dexamethasone (Dex), (iii) this inhibition could be relieved by RU486 co-administration, and also if it could be correlated with GR and corepressor binding to their putative IR nGREs.

For 9 of the above “IR1” genes, the repressing activity of their putative IR1 nGREs was tested in vitro using the luciferase assay. In all cases, and irrespective of the tissue pattern of GC-induced repression, these putative IR1 nGREs “repressed” luciferase activity (FIG. 13), indicating that these IR1 nGREs elements are bona fide IR1 nGREs. Importantly, upon GC treatment in vivo, there was a tight correlation between repression of a given gene in a given tissue (and its relief by RU co-administration), and GR and corepressor association with the IR1 nGRE of that gene in that tissue. In contrast there was no GR and corepressor association with the IR1 element of BMP3 gene which is not “repressed” in either tissue.

The “IR0” genes (Table 4) and “IR2” genes (Table 4) were also cell-specifically or non-cell-specifically expressed and repressed in the three tissues. The GC-induced repressing potential of putative IR nGREs of some of the selected “IR0” and “IR2” genes was tested using the luciferase assay, and it was found that they exhibited a repressing activity, irrespective of their activity in vivo (not shown). Upon Dex treatment, a decrease in gene expression in a given tissue always correlated with GR and corepressor association with the IR0 or IR2 element in that tissue, and in all cases the GC-induced repression was relieved by co-administration of RU. It can be concluded that, as expected from the in vitro luciferase assays, not only IR1, but also IR0 and IR2 nGREs can efficiently mediate the cis-acting GC agonist-induced repressing activity of the GR, in vivo.

TABLE 4
(A) Distribution of mouse and human IR0, IR1 and IR2 nGREs

| | IR0 | IR1 | IR2 |
|---|---|---|---|
| mouse genes containing a putative nGRE | 1105 | 3303 | 3949 |
| human genes containing a putative nGRE | 1169 | 4281 | 5443 |
| mouse and human orthologue genes containing identical IR nGREs | 51 | 379 | 566 |

(B) Repression, upon mouse treatment with a GC agonist, of genes containing IR0, IR1 and IR2
nGREs conserved in their human orthologues.

| IR0 nGRE | | | | | | | | | |
|---|---|---|---|---|---|---|---|---|---|
| containing | | | | Intestinal | | | | | |
| Genes | Epidermis | | | epithelium | | | Liver | | |
| ADAMTS19 | | | + | | | + | | | |
| AIP | | ♦ | + | | ♦ | + | | ♦ | + |
| ANO4 | | | | | ♦ | + | | | + |
| CCND1 | | | + | | | + | | | + |
| CITED1 | | | | | ♦ | + | | ♦ | + |
| DDI1 | | ♦ | + | | | | | | + |
| DLAT | | ♦ | + | | ♦ | + | | ♦ | + |
| FBXL18 | | ♦ | + | | ♦ | + | | ♦ | + |
| FSD2 | | ♦ | + | | | + | | | |
| GPIHBP1 | | ♦ | + | | ♦ | + | | ♦ | + |
| HCST | | ♦ | + | | | + | | | + |
| INHBE | | | | | | | | ♦ | + |
| KLF5 | | ♦ | + | | ♦ | + | | | + |
| LAG3 | | ♦ | + | | ♦ | + | | | + |
| MAPK4 | | | | | ♦ | + | | | + |
| MTG1 | | ♦ | + | | ♦ | + | | ♦ | + |
| NMYC | | | + | | | + | | ♦ | + |
| NDUFA4L2 | | | + | | ♦ | + | | | + |
| NEU1 | | ♦ | + | | ♦ | + | | | |
| PPIL5 | | | + | | ♦ | + | | | |
| PTMS | | ♦ | + | | ♦ | + | | ♦ | + |
| SLC25A5 | | ♦ | + | | ♦ | + | | | + |
| SP9 | | | | | ♦ | + | | | |
| SPARC | | | + | | ♦ | + | | | + |
| TCF25 | | ♦ | + | | ♦ | + | | ♦ | + |
| TRPM7 | | ♦ | + | | ♦ | + | | | + |
| ZBED4 | | ♦ | + | | ♦ | + | | ♦ | + |
| ZFP113 | | ♦ | + | | ♦ | + | | ♦ | + |
| ZFP467 | | ♦ | + | | ♦ | + | | | + |
| ZFP784 | | ♦ | + | | ♦ | + | | ♦ | + |
| | repressed | not repressed | expressed | repressed | not repressed | expressed | repressed | not repressed | expressed |
| IR1 nGRE | | | | | | | | | |
| containing | | | | Intestinal | | | | | |
| genes | Epidermis | | | epithelium | | | Liver | | |
| ADRA2A | | | + | | ♦ | + | | ♦ | + |
| AGPAT2 | | ♦ | + | | ♦ | + | | ♦ | + |
| AKAP6 | | ♦ | + | | | + | | | + |
| ARHGAP23 | | ♦ | + | | | + | | | + |
| ATN1 | | ♦ | + | | ♦ | + | | | + |
| BCL2L1 | | ♦ | + | | ♦ | + | | | + |
| BEST2 | | ♦ | + | | | | | | |
| BMP3 | | ♦ | + | | ♦ | + | | ♦ | + |
| BTG2 | | | | | | + | | | + |
| B4GALT2 | | ♦ | + | | | + | | | + |
| CACNA2D3 | | ♦ | + | | | | | | |
| CADM4 | | ♦ | + | | ♦ | + | | ♦ | + |
| CDH11 | | | + | | ♦ | + | | ♦ | + |
| CDK9 | | ♦ | + | | ♦ | + | | ♦ | + |
| CTSA | | ♦ | + | | ♦ | + | | ♦ | + |
| CYP26A1 | | | + | | | | | | + |
| DYNLT3 | | ♦ | + | | ♦ | + | | ♦ | + |
| FBLN2 | | | + | | | + | | | + |
| GBA2 | | | | | | + | | | + |
| GNG10 | | ♦ | + | | | + | | | + |
| GPR108 | | ♦ | + | | ♦ | + | | ♦ | + |
| GPR153 | | ♦ | + | | ♦ | + | | ♦ | + |
| HSD11B2 | | | + | | | | | ♦ | + |
| JUND | | ♦ | + | | | + | | | + |
| KLC3 | | ♦ | + | | ♦ | + | | ♦ | + |
| KRT14 | | | + | | | | | | |
| LRRC | | ♦ | + | | ♦ | + | | ♦ | + |
| MT2 | | ♦ | + | | | + | | ♦ | + |
| MTHFR | | ♦ | + | | ♦ | + | | ♦ | + |
| MXI1 | | ♦ | + | | ♦ | + | | ♦ | + |
| NKRF | | ♦ | + | | ♦ | + | | | + |
| PRKCB | | | + | | | + | | | + |
| RDHI1 | | | | | | + | | | + |
| SCP2 | | ♦ | + | | ♦ | + | | ♦ | + |
| SGK3 | | | | | | | | ♦ | + |
| SOCS3 | | | | | ♦ | + | | | + |
| SREPF1 | | ♦ | + | | ♦ | + | | ♦ | + |
| SSTR4 | | ♦ | + | | ♦ | + | | ♦ | + |
| STAB1 | | ♦ | + | | ♦ | + | | ♦ | + |
| STRA13 | | | + | | | + | | | + |
| TNFRSF19 | | | + | | | + | | | + |
| USF1 | | ♦ | + | | | + | | | + |
| | repressed | not repressed | expressed | repressed | not repressed | expressed | repressed | not repressed | expressed |
| IR2 nGRE | | | | | | | | | |
| containing | | | | Intestinal | | | | | |
| genes | Epidermis | | | epithelium | | | Liver | | |
| CCR10 | | | | | | + | | ♦ | + |
| COL4A3BP | | ♦ | + | | ♦ | + | | ♦ | + |
| DPAGT1 | | | + | | ♦ | + | | ♦ | + |
| ELMOD3 | | ♦ | + | | ♦ | + | | ♦ | + |
| ERP27 | | | | | ♦ | + | | ♦ | + |
| FKBP2 | | ♦ | + | | | + | | | + |
| FLOT2 | | | + | | | + | | | + |
| FOXJ1 | | | | | | + | | ♦ | + |
| FOXQ1 | | ♦ | + | | | + | | ♦ | + |
| FSTL1 | | | + | | ♦ | + | | | + |
| GAS6 | | | + | | | + | | | |
| GAS2L2 | | | | | | + | | | |
| GGT1 | | ♦ | + | | | + | | ♦ | + |
| GIT2 | | ♦ | + | | ♦ | + | | | + |
| GKN1 | | | + | | | | | | |
| GLU1 | | ♦ | + | | ♦ | + | | | + |
| GOT1 | | ♦ | + | | ♦ | + | | ♦ | + |
| GPR68 | | | | | ♦ | + | | | + |
| GPR97 | | ♦ | + | | | + | | ♦ | + |
| GRASP | | ♦ | + | | | + | | ♦ | + |
| GUK1 | | | | | ♦ | + | | | + |
| HSPB2 | | ♦ | + | | ♦ | + | | ♦ | + |
| HSPB3 | | | | | ♦ | + | | ♦ | + |
| ISL2 | | | | | | + | | | |
| ITPKA | | | + | | ♦ | + | | | + |
| MUC20 | | | | | ♦ | + | | ♦ | + |
| POMGNT1 | | ♦ | + | | | + | | ♦ | + |
| PRKRA | | ♦ | + | | ♦ | + | | ♦ | + |
| RDH5 | | | | | | + | | | + |
| RORA | | ♦ | + | | | + | | | + |
| SLC25A34 | | | | | ♦ | + | | | + |
| SLC2A8 | | ♦ | + | | ♦ | + | | | + |
| SOCS1 | | | | | ♦ | + | | ♦ | + |
| TGM6 | | ♦ | + | | ♦ | + | | | |
| TNFRSF25 | | | | | ♦ | + | | ♦ | + |
| | repressed | not repressed | expressed | repressed | not repressed | expressed | repressed | not repressed | expressed |

RNA isolated from 18 hr-Dex-treated WT mice epidermis, intestinal epithelium and liver was analyzed by Q-RT-PCR.
Genes, for which no signal could be detected in tissue samples from vehicle treated mice after 60 cycles of amplification, were considered to be not expressed in that tissue.
(+) indicates that the gene was expressed in that tissue while “Repressed” denotes statistically (p < 0.05) significant decrease in transcript levels after Dex treatment.

D) Differential Effects of the GRdim Mutation and RU486 Treatment on GC-Induced “Tethered”- and IR nGRE-Mediated Transrepressions.

The GRdim mutation does not affect GC-induced NFκB and AP1-mediated “tethered” transrepression, whereas it impairs (+)GRE transactivation (Tuckermann et al., 1999). In contrast, we found that Dex-induced IR nGRE-mediated gene repression was abolished by this mutation (data not shown). Expression of “IR0 nGRE” CCND1, “IR1 nGRE” PRKCB and “IR2 nGRE” FSTL1 genes, was repressed by Dex treatment in WT epidermis, but not in GRdim mutant. Note that epidermis was treated as indicated with TPA, to activate NFκB and AP1 factors. As expected, the repression of the Cox2 gene that contains NFκB and AP1 binding sites, was not impaired in mutant epidermis, while the (+)GRE-containing GPX3 gene was not GC-induced (FIG. 14). Accordingly, ChIP assays showed that Dex-induced repressing complexes were formed on IR nGREs of CCND1, PRKCB and FSTL1 genes of WT but not of GRdim mice, while no activating complex was formed on the GPX3 (+)GRE gene in Dex-treated epidermis of GRdim mutants (not shown), and repressing complexes were assembled, upon epidermis Dex treatment, on the Cox2 NFκB/AP1-containing region in both WT and dim mutants (not shown).

Importantly, the effect of Dex treatment was reverted by excess RU co-treatment in the case of IR nGRE-mediated transrepression, whereas RU had little effect on NFκB/AP1-mediated “tethered” transrepression (FIG. 14). Similar results concerning the differential RU effects were obtained when NFκB and AP1 factors were activated in MLE12 mouse cells by addition of IL-1β, instead of TPA treatment (not shown).

E) Failure of “Dissociated GCs” to Prevent Undesirable Side Effects of Corticoid Therapy could be Due to IR nGREs-Mediated Transrepression.

The present results led to posit that GC-induced transcriptional repression of IR nGRE-containing genes could contribute to the GC undesirable effects.

IL-1β-“activated” A549 cells transfected with pGL4- and pGL3-based reporter plasmids were used to examine the activities exhibited in vitro by RU24858 for (i) “tethered” transrepression (FIGS. 2 and 3, PGL4-NFκB and PGL4-AP1 vector), and (ii) (+)GRE-mediated transactivation (FIG. 5, PGL3(VDRE) (+)GRE vector). As expected from its “dissociated” profile in vitro, RU24858 was almost as efficient as Dex at repressing IL1β-induced activation of transcription by NFκB (FIG. 15A, left panel) and AP1 (FIG. 15A, right panel). Accordingly, RU24858 was as efficient as Dex at recruiting a repressing complex “tethered” to NFκB bound to its cognate element in the PGL4-NFκB vector (not shown). In contrast, RU24858 was much less efficient than Dex at inducing transactivation of (+)GRE pGL3 luciferase plasmid (not shown).

The “activity profile” of RU24858 was also investigated in vivo. Unlike Dex, a topical RU24858 treatment did not activate GC-dependent expression of the (+)GRE-containing GPX3 gene in epidermis, nor of the GGT1 and ERP27 genes in liver (not shown), and did not induce assembly of an activating complex on their (+)GRE (data not shown). In contrast, RU24858 was as efficient as Dex at down-regulating, through NFκB-mediated “tethered” transrepression, genes of which the skin expression was enhanced by topical TPA treatment (not shown). Most interestingly, and as in vitro, RU24858 was also as efficient as Dex at inducing transrepression of IR0 (CCND1), IR1 (TSLP, CYP26A1, K14, PRKCB) and IR2 (DPAGT1) nGRE-containing genes (FIG. 15B), through recruitment of GR-SMRT/NCoR repressing complexes on nGRE regions (data not shown).

Long-term treatments with GCs generate numerous debilitating effects. An ontology search revealed that the known or putative functions of almost 15% of IR nGRE-containing orthologue genes could possibly be implicated in physiological homeostatic processes leading to side-effects upon GC therapy. Moreover, there is evidence that repression of expression of a number of these latter genes could actually be instrumental to the generation of defects subsequent to GC administration, either because their expression is known to be decreased upon GC treatment, and/or because their decreased expression is known to generate defects related to those produced by GC-therapy. Note, however, that many of the NFκB and AP1 binding site-containing genes that encode regulatory components (e.g. cytokines) of the immune system, also contain IR nGREs, while genes encoding anti-apoptotic proteins (Bcl2 and Bcl-XL), as well as mitogenic proteins involved in cell cycle progression at the G1/S phase (Cyclin D1 and CDK4) can also be GC-transrepressed via IRnGREs (Table 5).

TABLE 5
A. IR nGRE-containing genes whose GC-induced transrepression could generate side effects related
to those produced by GC therapy

| Debilitating side | | | | | |
|---|---|---|---|---|---|
| effects upon GC | Gene | | | | |
| therapy | symbol | Gene name | a | b | References |
| Skin atrophy, | Krt 14, Krt 5 | Keratin 14 (IR1), Keratin 5 (IR1) | + | − | Ramot et al., 2009 |
| bruising, thinning, | TGFβ1 | Transforming growth factor beta 1 precursor (IR1) | + | − | Frank et al., 1996 |
| brittle skin, | Smad4 | SMAD family member 4 (IR2) | − | + | Chen et al., 2000 |
| disturbed wound | Tnc | Tenascin C (IR2) | + | − | Fassler et al., 1996 |
| healing | Trpv3 | Transient receptor potential cation channel | − | + | Cheng et al., 2010 |
| (Schacke et al, | | subfamily V member 3 (IR2) | | | |
| 2002) | Ccnd1 | Cyclin D1 (IR0) | + | + | |
| | Cdk4 | Cyclin-dependent kinase 4 (IR2) | + | + | Rogatsky et al., 1999 |
| Impaired skeletal | Tnfrsf11b | Osteoprotegerin (IR2) | + | + | Sasaki et al., 2001 |
| growth and | Bcl2 | Bcl-2 (IR1) | + | − | Mocetti et al., 2001 |
| osteoporosis | Bcl2l1 | Bcl-XL (IR1) | + | − | Lu et al., 2007 |
| (Schacke et al., | TGFβ1 | Transforming growth factor beta 1 precursor (IR1) | − | + | Geiser et al., 1998 |
| 2002, Kleiman and | Smad 4 | SMAD family member 4 (IR2) | − | + | Tan et al., 2007 |
| Tuckermann, 2007) | Ghr | Growth hormone receptor (IR1) | + | + | Gevers et al., 2002 |
| | Gnas | Adenylate cyclase stimulating G-alpha protein (IR1) | − | + | Weinstein et al., 2004 |
| | Wnt5a | Wingless-related MMTV; integration site 5A (IR1) | − | + | Yang et al., 2003 |
| | Ahsg | Alpha-2-HS-glycoprotein precursor (IR0) | − | + | Szweras et al., 2002 |
| | Col11a2 | Collagen, type XI, alpha 2 chain precursor (IR2) | − | + | Li et al., 2001 |
| Hyperglycemia and | Ins | Insulin precursor (IR1, IR2) | + | + | Delaunay et al., 1997, |
| Diabetes (Schacke | Insr | Insulin receptor (IR1) | + | + | Caro and Amatruda., 1982, |
| et al, 2002, Kleiman | | | | | |
| and Tuckermann, | | | | | |
| 2007) | | | | | |
| Muscle | ctnnb1 | Beta-catenin (IR1) | + | + | Schakman et al.; 2008b |
| atrophy/Myopathy | Akt1 | Protein kinase 8 (IR2) | − | + | Schakman et al., 2008a |
| (Schakman et al., | Tpm2 | Tropomysin beta chain (IR1) | − | + | Ochala et al., 2007 |
| 2008a) | | | | | |
| Impaired HPA axis, | Mc2r | ACTH receptor (IR1) | + | + | Chida et al., 2007 |
| Adrenal | Mrap | ACTH receptor accessory protein (IR1) | + | + | Metherell et al., 2005 |
| insufficiency | | | | | |
| (Schacke et al., | | | | | |
| 2002) | | | | | |
| Circadian rhythm | Clock | Circadian locomotor output cycle kaput protein (IR1) | − | + | Roybal et al., 2007 |
| disorder, metabolic | Nr1d1 | Reverba (IR1) | + | − | Torra et al., 2000, Preitner et |
| syndrome, bipolar | | | | | al., 2002 |
| disorder and mania | Rora | RORα (IR2) | + | − | This study |
| (Bechtold et al., | | | | | |
| 2010, Duez and | | | | | |
| Staels, 2008) | | | | | |
| Anxiety and | Ucn2 | Urocortin 2 (IR1) | + | + | Chen et al., 2004, 2006; |
| depression | | | | | Neufeld-Cohen et al., 2010 |
| (Schacke et al., | Crhr-2 | Corticotropin releasing hormone receptor 2 (IR1) | − | + | Bale et al., 2000 |
| 2002) | | | | | |
| Hypertension | Hsd11b2 | Corticosteroid 11-beta-dehydrogenase isozyme 2 | + | + | Stewart et al., 1996 |
| (Schacke et al, | | (11β-HSD2) (IR1) | | | |
| 2002) | | | | | |

a. Genes whose expression is known to be decreased upon GC treatment.
b. Genes, whose decreased expression is known to generate effects related to those produced by GC
treatment.
(IR0), (IR1) and (IR2) indicate the type of nGRE motif present in that gene.

B. IR nGRE-containing genes involved in GC-anti-inflammatory therapy

| Gene | | Gene | |
|---|---|---|---|
| symbol | Gene name* | symbol | Gene name* |
| C1qb | Complement C1q subcomponent subunit B Precursor (IR2) | Il16 | Interleukin-16 Precursor (IR1) |
| C1ql1 | C1q-related factor Precursor (IR1) | Il17rb | Interleukin-17 receptor b (IR1) |
| C3 | Complement C3 Precursor (IR1) | Il11ra1 | Interleukin-11 receptor (IR1) |
| Cfd | Complement factor D Precursor (IR1) | Il17b | Interleukin-17b Precursor (IR1) |
| Il6 | Interleukin-6 Precursor (IR1) | Il17f | Interleukin-17f Precursor (IR1) |
| Il20 | Interleukin-20 Precursor (IR2) | Il28a | Interleukin-28a Precursor (IR1) |
| Ccr10 | C-C chemokine receptor type 10 (IR2) | Il28b | Interleukin-28b Precursor (IR1) |
| Stat3 | Signal transducer and activator of transcription 3 (IR2) | Il24 | Interleukin-24 Precursor (IR1) |
| Nfatc1 | Nuclear factor of activated T-cells, cytoplasmic 1 (IR1) | Il34 | Interleukin-34 Precursor (IR1) |
| Il8ra | Interleukin-8 receptor a (IR1) | Il1rn | Interleukin-1 receptor antagonist protein |
| Il12rb1 | Interleukin-12 receptor b1 (IR1) | | Precursor (IR2) |
| Il17ra | Interleukin-17 receptor a (IR1) | Il22ra1 | interleukin-22 receptor a1 (IR1) |
| | | TSLP | Thymic Stromal Lymphopoietin (IR1) |

*All of these genes also contain NFκB and AP1 binding sites.

### BIBLIOGRAPHY

Bechtold, D. A., Gibbs, J. E., and Loudon, A. S. (2010). Circadian dysfunction in disease. Trends Pharmacol. Sci. 31, 191-198.
Belvisi, M. G., et al. (2001). J. Immunol. 166, 1975-1982,
Carey, M., et al. (2001). Nex York Cold Spring Harbor Laboratory Press.
Chrousos, G. P. (2009). Nat. Rev. Endocrinol. 5, 374-381.
De Bosscher, K., and Haegeman, G. (2009). Mol. Endocrinol. 23, 281-291.
Delaunay, F., et al. (1997). J. Clin. Invest. 100, 2094-2098.
Dostert, A., and Heinzel, T. (2004). Curr. Pharm. Des. 10, 2807-2816.
Duez, H., and Stack, B. (2008) FEBS Lett. 582, 19-25.
Elbe-Burger, A., et al. (1997) Immunology Methods Manual, ed. Lefkovits, pp 1490-1501.
Flores-Langarica, A., et al. (2005). Proc. Natl. Acad. Sci. USA 102, 19039-19044.
Green, S., et al. (1988). Nuc. Acid Res. 16, 369.
Gross, K. L., and Cidlowski J. A. (2008). Trends Endocrinol. Metab. 19, 331-339.
Huang, Da. W., et al. (2009). Nat. Protoc. 4, 44-57,
Karin, M. (11998). Cell 93, 487-490.
Kassel, O., and Herrlich, P. (2007). Mol. Cell. Endocrinol. 275, 13-29.
Kleiman, A., and Tuckerman, J. P. (2007). Glucocorticoid receptor action in beneficial and side effects of steroid therapy: Lessons from conditional knockout mice, Mol. Cell. Endocrinol. 275, 98-108.
Li, M., et al. (2005). Proc. Natl. Acad. Sci. USA 102, 14795-14800
Liu, Z. and Garrard, W. T. (2005). Mol. Cell. Biol. 25, 3220-3231.
Lonard, D. M., and O'Malley, B. W. (2007). Nuclear receptor coregulators: judges, juries and executioners of cellular regulation, Mol. Cell. 27, 691-700.
Meijsing, S. H., et al. (2009). Science 324, 407-410.
Nader, N., et al. (2010). Trends Endocrinol. Metab. 21, 277-286.
Ramot, Y., et al. (2009). Bioessays 31, 389-399.
Reddy, T. E., et al. (2009). Genome Res. 19, 2163-2171.
Reichardt, H. M., et al. (11998). Cell 93, 531-41.
Rhen, T., and Cidlowski, J. A. (2005). N. Engl. J. Med. 353, 1711-23.
Ricketson, D., et al., (2007). J. Mol. Biol. 368, 729-741
Sambrook, J., et al. (1989). Molecular Cloning: A Laboratory Manual, 2nd Ed. (New York: Cold
Schake, H., Docke, W. D., and Asadullah, K. (2002). Mechanisms involved in the side effects of glucocorticoids. Pharmacol. Ther. 96, 23-43.
Schakman, O., Gilson, H., and Thissen, J. P. (2008a). Mechanisms of glucocorticoid-induced myopathy. J. Endrocrinol. 197, 1-10. Spring Harbor Laboratory Press).
Stewart, P. M., et al. (1996). Lancet 347, 88-91
Tuckermann, J. P. et al. (1999). J. Cell Biol. 147, 1365-1370.
Vaisanen, S., Dunlop, et al. (2005). J. Mol. Biol. 350, 65-77.
Vayssiere, B. M., et al. (1997). Mol. Endocrinol. 11, 1245-1255.
Wang J. C., Derynck et al. (2004). Proc. Natl. Acad. Sci. USA 101, 15603-15608.

1. A process for selecting a molecule exhibiting anti-inflammatory activities of glucocorticoids (GCs), via glucocorticoid receptor (GR)-dependent tethered indirect transrepression, and having reduced GC-dependent IR nGRE-mediated direct transrepression activity, comprising the following steps:
i) providing at least one candidate molecule,
ii) testing if said candidate molecule:
a) significantly induces GR-dependent tethered indirect transrepression of the transcription of at least one gene containing among its promoter elements at least one NFκB or one AP1 DNA binding sequence (DBS),
b) significantly induces GR-dependent direct transrepression of the transcription of at least one gene containing among its promoter elements an IR-type negative element (IR nGRE DBS),
c) significantly induces GR-dependent transactivation of the transcription of at least one gene containing among its promoter elements a (+) GRE DBS,
iii) selecting said candidate molecule:
if it significantly transrepresses the transcription of said gene in step a), but it does not significantly transrepress the transcription of said gene in step b), or
if it significantly transrepresses the transcription of said gene in step a), but does not significantly transrepress the transcription of said gene in step b) and does not significantly increase the transactivation of the transcription of said gene in step c),
wherein said IR nGRE DBS is chosen in the group consisting of: sequence SEQ ID NO:1 (IR1 nGRE), SEQ ID NO:2 (IR0 nGRE), SEQ ID NO:3 (IR2 nGRE) and tolerable variants thereof,
wherein said NFκB DBS has the sequence SEQ ID NO: 4, SEQ ID NO:5 or SEQ ID NO:6, or variants thereof,
wherein said AP1 DBS has the sequence SEQ ID NO:7, SEQ ID NO:8 or variants thereof, and
wherein said (+) GRE DBS has the sequence SEQ ID NO:9 or SEQ ID NO:10 or variants thereof.

2. The process according to claim 1, comprising at least the following in vitro steps, in cultured GR-expressing cells:
a) providing at least one candidate molecule,
b) providing a first vector comprising at least one NFκB or one AP1 DBS operatively coupled to a reporter gene and a promoter thereof active in said cells,
c) providing a second vector comprising an IR nGRE DBS operatively coupled to a reporter gene and a promoter thereof active in said cells,
d) providing a third vector comprising a (+) GRE DBS sequence operatively coupled to a reporter gene and a promoter thereof active in said cells,
e) selecting conditions wherein NFκB and/or AP1 factors are activated in step b),
f) selecting conditions wherein said reporter genes of steps b) and c) are significantly expressed and significantly repressed by GCs, and wherein the expression of said reporter gene of step d) is significantly increased by the addition of GC,
g) measuring in said GR-expressing cells the level of expression of each reporter gene in the presence and absence of said candidate molecule, and under conditions selected in steps e) and f),
h) selecting said candidate molecule:
if it significantly transrepresses the expression of said reporter gene of step b), but does not significantly transrepress the expression of said reporter gene of step c), or
if it significantly transrepresses the expression of said reporter gene of step b), but does not significantly transrepress the expression of said reporter gene of step c), and does not significantly increase the expression of said reporter gene of step d).

3. The process according to claim 2, wherein said step g) comprises the following sub-steps:
g0) measuring the expression level E0 of each reporter gene in the absence of said candidate molecule and under conditions wherein the expression of said reporter genes of steps b) and c) is significantly repressed by GC, whereas the expression of said reporter gene of step d) is significantly increased by the addition of GC,
g1) measuring the expression level E1 of each reporter gene in the presence of said candidate molecule, all other conditions being the same as in step g0),
and wherein, in said step h), said candidate molecule is selected if:
E1 is significantly lower than E0 for said reporter gene of step b), and E1 is not significantly lower than E0 for said reporter gene of step c), or
E1 is significantly lower than E0 for said reporter gene of step b), and E1 is not significantly lower than E0 for said reporter gene of step c), and E1 is not significantly higher than E0 for said reporter gene of step d).

4. The process according to claim 1, wherein said tolerable variant is an IR nGRE DBS derived by at least one base pair change from the sequence SEQ ID NO:1 (IR1 nGRE), SEQ ID NO:2 (IR0 nGRE), or SEQ ID NO:3 (IR2 nGRE), and has a GR-dependent direct transrepression activity.

5. The process according to claim 1, wherein said tolerable variant of SEQ ID NO:1 (IR1 nGRE) is chosen in the group consisting of: SEQ ID NO:11 to 27, and said tolerable variant of SEQ ID NO:3 (IR2 nGRE) is chosen in the group consisting of: SEQ ID NO:28 to 48.

6. The process according to claim 2, wherein said first vector is SEQ ID NO:55 (PGL4-AP1) or SEQ ID NO:56 (PGL4-NFκB), wherein said second vector is SEQ ID NO: 49 (PGL3 (SV40/VDRE) IR1 nGRE), SEQ ID NO:50 (PGL3 (VDRE) IR1 nGRE), SEQ ID NO:51 (PGL3(VDRE) IR0 nGRE), or SEQ ID NO:52 (PGL3(VDRE) IR2 nGRE) and wherein said third vector is SEQ ID NO:53 (PGL3 (SV40/VDRE) (+)GRE) or SEQ ID NO:54 (PGL3 (VDRE) (+) GRE).

7. The process according to claim 1, comprising the following steps in GR-expressing cells:
a) providing at least one candidate molecule,
b) choosing at least one proinflammatory genomic gene which is known to be transrepressed by GC, and containing among its promoter elements at least one NFκB and/or one AP1 DBS, said gene being significantly expressed in said cells,
c) choosing at least one genomic gene which is known to be transrepressed by GC, and containing among its promoter elements at least one IR nGRE DBS, said gene being significantly expressed in said cells,
d) choosing at least one genomic gene containing among its promoter elements at least one (+) GRE DBS, the expression of said gene being significantly increased by the addition of GC in said cells,
e) selecting conditions wherein NFκB and/or AP1 factors are activated in step b), and selecting conditions wherein said reporter genes of steps b) and c) are significantly expressed and significantly repressed by GCs, and wherein the expression of said reporter gene of step d) is significantly increased by the addition of GC,
f) measuring the expression level E0 of each gene in said cells in the absence of said candidate molecule, under conditions selected in step e),
g) providing said candidate molecule to said cells and measuring the expression level E1 of each gene in the presence of said candidate molecule, all other conditions being the same as in step f),
h) selecting said candidate molecule if:
E1 is significantly lower than E0 for said gene of step b), and E1 is not significantly lower than E0 for said gene of step c), or
E1 is significantly lower than E0 for said gene of step b), and E1 is not significantly lower than E0 for said gene of step c), and E1 is not significantly higher than E0 for said gene of step d).

8. The process according to claim 7, further comprising the step of providing concomitantly to said candidate molecule the antiglucocorticoid RU486 and measuring the expression level E2 of one gene chosen in step b) in the presence of said candidate molecule and said antiglucocorticoid, all other conditions being the same as in step f), and wherein said candidate molecule is selected if E2 is significantly lower than E0 for said gene of step b).

9. The process according to claim 7, wherein said GR-expressing cells are in vitro cultured cells, preferably human lung epithelial carcinoma cells A549, and wherein said gene of step b) is chosen from the COX2 or MMP13 genes, said gene of step c) is chosen from the BCL3, BHLHB2, ENC1, FGFR3, FOXa2, GEM and MAFK genes, and said gene of step d) is the GILZ gene.

10. The process according to claim 7, wherein said GR-expressing cells are obtained from a tissue of an animal, preferably a mouse, and wherein said gene of step b) is chosen in the group consisting of: the COX2, IL4, IL1β, TNFα, MMP13 and IL10 genes, said gene of step c) is chosen in the group consisting of: CCND1, CYP26A1, JUND, HSD11β2, PRKCB, K14, RDH11, STRA13, RORα and TNFRSF19 genes and said gene of step d) is chosen in the group consisting of: the GPX3, GGT1 and ERP27 genes.

11. The process according to claim 7, wherein said GR-expressing cells are:
a) epidermal cells of the skin of an animal, preferably a mouse, on which said candidate molecule has been administered topically without or with the antiglucocorticoid RU486, wherein said gene in step b) is chosen in the group consisting in: the COX2, IL4, IL1β, TNFα, MMP13 and IL10 genes, said gene of step c) is chosen in the group consisting in: the CCND1, CYP26A1, HSD11β2, PRKCB, K14, STRA13, and TNFRSF19, and said gene of step d) is the GPX3 gene,
b) cells obtained from the liver of an animal, preferably a mouse, on which said candidate molecule has been administered without or with the antiglucocorticoid RU486, wherein said gene in step b) is the COX2 gene, said gene of step c) is chosen in the group consisting in: the CCND1, JUND, PRKCB, RDH11, STRA13, RORα and TNFRSF19 genes, and said gene of step d) is chosen in the group consisting of: the GPX3, GGT1 and ERP27 genes, or
c) cells obtained from the intestinal epithelium of an animal, preferably a mouse, on which said candidate molecule has been administered without or with the antiglucocorticoid RU486, wherein said gene in step b) is chosen in the group consisting in: the COX2 and IL1β genes, said gene of step c) is chosen in the group consisting in: the CCND1, JUND, PRKCB, RDH11, STRA13, RORα and TNFRSF19 genes, and said gene of step d) is the GPX3 gene.

12. Use of an IR nGRE DBS present in recombinant vectors and animal genomes for identifying a molecule exhibiting anti-inflammatory activities of GCs and having reduced GC-dependent IR nGRE-mediated direct transrepression activity.

13. The use according to claim 12, wherein said IR nGRE DBS is chosen in the group consisting of: sequence SEQ ID NO:1 (IR1 nGRE), SEQ ID NO:2 (IR0 nGRE), SEQ ID NO:3 (IR2 nGRE), or tolerable variants thereof, said tolerable variant of SEQ ID NO:1 (IR1 nGRE) being preferably chosen in the group consisting of: SEQ ID NO:11 to 27, and said tolerable variant of SEQ ID NO:3 (IR2 nGRE) being preferably chosen in the group consisting of: SEQ ID NO:28 to 48.

14. Use of the antiglucocorticoid RU486 or of any GR antagonist having an IR nGRE-mediated transrepression activity similar to that of the antiglucocorticoid RU486, for identifying a molecule exhibiting anti-inflammatory activities of GCs and having reduced GC-dependent IR nGRE-mediated direct transrepression activity.

15. An isolated IR nGRE DNA binding site, chosen in the group consisting of: sequence SEQ ID NO:1 (IR1 nGRE), SEQ ID NO:2 (IR0 nGRE), SEQ ID NO:3 (IR2 nGRE), or tolerable variants thereof.

16. An isolated vector containing an IR nGRE DBS as defined in claim 1, said vector being preferably chosen in the group consisting of: SEQ ID NO: 49 (PGL3 (SV40/VDRE) IR1 nGRE), SEQ ID NO:50 (PGL3 (VDRE) IR1 nGRE), SEQ ID NO:51 (PGL3(VDRE) IR0 nGRE), and SEQ ID NO:52 (PGL3(VDRE) IR2 nGRE).


SEQUENCE LISTING (56 sequences)

SEQ ID NO: 1 - DNA, 10 bp, artificial
  IR1 nGC Response Element (GRE)
ctccnggaga

SEQ ID NO: 2 - DNA, 9 bp, artificial
  IR0 GRE
ctccggaga

SEQ ID NO: 3 - DNA, 11 bp, artificial
  IR2 GRE
ctccnnggaga

SEQ ID NO: 4 - DNA, 10 bp, artificial
  NFkB DNA Binding Site (DBS)
gggrnnyycc

SEQ ID NO: 5 - DNA, 10 bp, artificial
  NFkB (DBS)
gggaatttcc

SEQ ID NO: 6 - DNA, 10 bp, artificial
  NFkB DBS
gggactttcc

SEQ ID NO: 7 - DNA, 7 bp, artificial
  AP1 DBS
tgagtca

SEQ ID NO: 8 - DNA, 7 bp, artificial
  AP1 DBS
tgactca

SEQ ID NO: 9 - DNA, 15 bp, artificial
  (+)GRE DBS consensus
rgnacannntgtnct

SEQ ID NO: 10 - DNA, 15 bp, artificial
  (+) GRE DBS
tgtacaggatgttct

SEQ ID NO: 11 - DNA, 10 bp, artificial
  tolerable variants of IR nGRE DBS
atccaggaga

SEQ ID NO: 12 - DNA, 10 bp, artificial
  tolerable variants of IR nGRE DBS
gtccaggaga

SEQ ID NO: 13 - DNA, 10 bp, artificial
  tolerable variants of IR nGRE DBS
ttccaggaga

SEQ ID NO: 14 - DNA, 10 bp, artificial
  tolerable variants of IR nGRE DBS
caccaggaga

SEQ ID NO: 15 - DNA, 10 bp, artificial
  tolerable variants of IR nGRE DBS
ccccaggaga

SEQ ID NO: 16 - DNA, 10 bp, artificial
  tolerable variants of IR nGRE DBS
ctgcaggaga

SEQ ID NO: 17 - DNA, 10 bp, artificial
  tolerable variants of IR nGRE DBS
cttcaggaga

SEQ ID NO: 18 - DNA, 10 bp, artificial
  tolerable variants of IR nGRE DBS
ctcgaggaga

SEQ ID NO: 19 - DNA, 10 bp, artificial
  tolerable variants of IR nGRE DBS
ctctaggaga

SEQ ID NO: 20 - DNA, 10 bp, artificial
  tolerable variants of IR nGRE DBS
ctccatgaga

SEQ ID NO: 21 - DNA, 10 bp, artificial
  tolerable variants of IR nGRE DBS
ctccaggcga

SEQ ID NO: 22 - DNA, 10 bp, artificial
  tolerable variants of IR nGRE DBS
ctccagggga

SEQ ID NO: 23 - DNA, 10 bp, artificial
  tolerable variants of IR nGRE DBS
ctccaggtga

SEQ ID NO: 24 - DNA, 10 bp, artificial
  tolerable variants of IR nGRE DBS
ctccaggaaa

SEQ ID NO: 25 - DNA, 10 bp, artificial
  tolerable variants of IR nGRE DBS
ctccaggata

SEQ ID NO: 26 - DNA, 10 bp, artificial
  tolerable variants of IR nGRE DBS
ctccaggagc

SEQ ID NO: 27 - DNA, 10 bp, artificial
  tolerable variants of IR nGRE DBS
ctccaggagg

SEQ ID NO: 28 - DNA, 11 bp, artificial
  tolerable variants of IR nGRE DBS
ctccacggaga

SEQ ID NO: 29 - DNA, 11 bp, artificial
  tolerable variants of IR nGRE DBS
atccacggaga

SEQ ID NO: 30 - DNA, 11 bp, artificial
  tolerable variants of IR nGRE DBS
gtccacggaga

SEQ ID NO: 31 - DNA, 11 bp, artificial
  tolerable variants of IR nGRE DBS
ttccacggaga

SEQ ID NO: 32 - DNA, 11 bp, artificial
  tolerable variants of IR nGRE DBS
caccacggaga

SEQ ID NO: 33 - DNA, 11 bp, artificial
  tolerable variants of IR nGRE DBS
ccccacggaga

SEQ ID NO: 34 - DNA, 11 bp, artificial
  tolerable variants of IR nGRE DBS
ctgcacggaga

SEQ ID NO: 35 - DNA, 11 bp, artificial
  tolerable variants of IR nGRE DBS
cttcacggaga

SEQ ID NO: 36 - DNA, 11 bp, artificial
  tolerable variants of IR nGRE DBS
ctcgacggaga

SEQ ID NO: 37 - DNA, 11 bp, artificial
  tolerable variants of IR nGRE DBS
ctctacggaga

SEQ ID NO: 38 - DNA, 11 bp, artificial
  tolerable variants of IR nGRE DBS
ctccaccgaga

SEQ ID NO: 39 - DNA, 11 bp, artificial
  tolerable variants of IR nGRE DBS
ctccactgaga

SEQ ID NO: 40 - DNA, 11 bp, artificial
  tolerable variants of IR nGRE DBS
ctccacgcaga

SEQ ID NO: 41 - DNA, 11 bp, artificial
  tolerable variants of IR nGRE DBS
ctccacggcga

SEQ ID NO: 42 - DNA, 11 bp, artificial
  tolerable variants of IR nGRE DBS
ctccacgggga

SEQ ID NO: 43 - DNA, 11 bp, artificial
  tolerable variants of IR nGRE DBS
ctccacggtga

SEQ ID NO: 44 - DNA, 11 bp, artificial
  tolerable variants of IR nGRE DBS
ctccacggaaa

SEQ ID NO: 45 - DNA, 11 bp, artificial
  tolerable variants of IR nGRE DBS
ctccacggaca

SEQ ID NO: 46 - DNA, 11 bp, artificial
  tolerable variants of IR nGRE DBS
ctccacggata

SEQ ID NO: 47 - DNA, 11 bp, artificial
  tolerable variants of IR nGRE DBS
ctccacggagc

SEQ ID NO: 48 - DNA, 11 bp, artificial
  tolerable variants of IR nGRE DBS
ctccacggagg

SEQ ID NO: 49 - DNA, 5557 bp, artificial
  PGL3(SV40/VDRE) IR1 nGRE
ggtaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaggtaccaggtcagagaggtcaaccactagaagacaggtcagagaggtcatgtggaaatcaaaggaaaggattttgttacattggccacagaaagtttctcatttcaccttctagtcttccatatcctaaagagtctagagggactttgtgaattcaatggctcaggccaataaagcaatctgaacatcatgtatactacaaataagtgagcaaagtggacagtcgtcacagtcatcacaaaccacacagcctatagcgtattacctggtcactatgccaaattgggactaatcaaaaagaatataaacaatgaaacaaggggttgaagcaatggttcagcaattaagagcacacaggcactgcgagctcttacgcgtgctagcccggggctgagctccaggagagtagggagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 50 - DNA, 5385 bp, artificial
  PGL3 VDRE IR1 nGRE
ggtaccaggtcagagaggtcaaccactagaagacaggtcagagaggtcatgtggaaatcaaaggaaaggattttgttacattggccacagaaagtttctcatttcaccttctagtcttccatatcctaaagagtctagagggactttgtgaattcaatggctcaggccaataaagcaatctgaacatcatgtatactacaaataagtgagcaaagtggacagtcgtcacagtcatcacaaaccacacagcctatagcgtattacctggtcactatgccaaattgggactaatcaaaaagaatataaacaatgaaacaaggggttgaagcaatggttcagcaattaagagcacacaggcactgcgagctcttacgcgtgctagcccggggctgagctccaggagagtagggagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 51 - DNA, 5384 bp, artificial
  PGL3(VDRE) IR0 nGRE
ggtaccaggtcagagaggtcaaccactagaagacaggtcagagaggtcatgtggaaatcaaaggaaaggattttgttacattggccacagaaagtttctcatttcaccttctagtcttccatatcctaaagagtctagagggactttgtgaattcaatggctcaggccaataaagcaatctgaacatcatgtatactacaaataagtgagcaaagtggacagtcgtcacagtcatcacaaaccacacagcctatagcgtattacctggtcactatgccaaattgggactaatcaaaaagaatataaacaatgaaacaaggggttgaagcaatggttcagcaattaagagcacacaggcactgcgagctcttacgcgtgctagcccggggctgagctccggagagtagggagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 52 - DNA, 5386 bp, artificial
  (PGL3(VDRE) IR2 nGRE
ggtaccaggtcagagaggtcaaccactagaagacaggtcagagaggtcatgtggaaatcaaaggaaaggattttgttacattggccacagaaagtttctcatttcaccttctagtcttccatatcctaaagagtctagagggactttgtgaattcaatggctcaggccaataaagcaatctgaacatcatgtatactacaaataagtgagcaaagtggacagtcgtcacagtcatcacaaaccacacagcctatagcgtattacctggtcactatgccaaattgggactaatcaaaaagaatataaacaatgaaacaaggggttgaagcaatggttcagcaattaagagcacacaggcactgcgagctcttacgcgtgctagcccggggctgagctccacggagagtagggagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 53 - DNA, 5562 bp, artificial
  PGL3(SV40/VDRE) (+) GRE
ggtaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaggtaccaggtcagagaggtcaaccactagaagacaggtcagagaggtcatgtggaaatcaaaggaaaggattttgttacattggccacagaaagtttctcatttcaccttctagtcttccatatcctaaagagtctagagggactttgtgaattcaatggctcaggccaataaagcaatctgaacatcatgtatactacaaataagtgagcaaagtggacagtcgtcacagtcatcacaaaccacacagcctatagcgtattacctggtcactatgccaaattgggactaatcaaaaagaatataaacaatgaaacaaggggttgaagcaatggttcagcaattaagagcacacaggcactgcgagctcttacgcgtgctagcccgggagaggatctgtacaggatgttctagatagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 54 - DNA, 5390 bp, artificial
  PGL3 VDRE (+) GRE
ggtaccaggtcagagaggtcaaccactagaagacaggtcagagaggtcatgtggaaatcaaaggaaaggattttgttacattggccacagaaagtttctcatttcaccttctagtcttccatatcctaaagagtctagagggactttgtgaattcaatggctcaggccaataaagcaatctgaacatcatgtatactacaaataagtgagcaaagtggacagtcgtcacagtcatcacaaaccacacagcctatagcgtattacctggtcactatgccaaattgggactaatcaaaaagaatataaacaatgaaacaaggggttgaagcaatggttcagcaattaagagcacacaggcactgcgagctcttacgcgtgctagcccgggagaggatctgtacaggatgttctagatagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 55 - DNA, 6036 bp, artificial
  PGL4 vector AP1
ggcctaactggccggtacctgagctcgctagctgagtcagtgagtcactgactcactgactcatgagtcagagatctggcctcggcggccaagcttagacactagagggtatataatggaagctcgacttccagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctcacggcttccctcccgaggtggaggagcaggccgccggcaccctgcccatgagctgcgcccaggagagcggcatggatagacaccctgctgcttgcgccagcgccaggatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 56 - DNA, 6059 bp, artificial
  PGL4 vector NFKB
ggcctaactggccggtacctgagctcgctagcgggaatttccggggactttccgggaatttccggggactttccgggaatttccgggaatttccagatctggcctcggcggccaagcttagacactagagggtatataatggaagctcgacttccagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctcacggcttccctcccgaggtggaggagcaggccgccggcaccctgcccatgagctgcgcccaggagagcggcatggatagacaccctgctgcttgcgccagcgccaggatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct